(19) United States
(12) Patent Application Publication
(10) Pub. No.: US 2026/0325167 A1
(43) Pub. Date: Oct. 22, 2026

(54) SYSTEMS AND METHODS FOR FIBER OPTIC RANGING AND IMAGING

(71) Applicants: University of Central Florida Research Foundation, Inc., Orlando, FL (US); Washington University, St. Louis, MO (US)

(72) Inventors: Guifang Li, Orlando, FL (US); Yonglin Huang, Orlando, FL (US); Song Hu, St. Louis, MO (US)

(21) Appl. No.: 19/462,848

(22) Filed: Jan. 28, 2026

Related U.S. Application Data
(60) Provisional application No. 63/750,679, filed on Jan. 28, 2025.

(51) Int. Cl. A61B 17/34; A61B 34/20; A61B 90/00; G01S 7/521; G01S 15/08

(52) U.S. Cl. CPC A61B 17/3403; A61B 34/20; A61B 90/06; G01S 7/521; G01S 15/08; A61B 2017/3413; A61B 2034/2063; A61B 2090/062

(57) ABSTRACT

A measurement system for ultrasonic ranging may include a fiber having at least two cores suitable for guiding light, an acoustic emitter on a distal end of a first core, wherein the acoustic emitter generates acoustic waves in response to incident light pulses, and an acoustic sensor on a distal end of a second core. A measurement system may include a gradient index lens, an acoustic emitter fabricated over a first portion of a face of the gradient index lens, and an acoustic sensor fabricated over a second portion of the face. A measurement system may include a gradient index lens and a multi-plane light converter configured to manipulate an intensity distribution and phase distribution of an input beam to a complex conjugate of a point spread function of the gradient index lens. Methods for ultrasonic ranging and photoacoustic imaging are also disclosed.

### CROSS-REFERENCE TO RELATED APPLICATIONS

The present application claims the benefit under 35 U.S.C. § 119(e) of U.S. Provisional Application No. 63/750,679 filed on Jan. 28, 2025, which is incorporated herein by reference in the entirety.

### TECHNICAL FIELD

The present disclosure relates generally to fiber optic probes for medical imaging and, more particularly, to systems and methods for depth ranging and photoacoustic imaging using gradient index lenses and multicore fibers suitable for, but not limited to, implementation within a needle.

### BACKGROUND

In traditional epidural and spinal injections, trained anesthetists utilize palpation techniques for guiding needles into the desired location in the patient. Anesthetists rely on training and tactile responses when guiding a syringe into position in the patient. However, the epidural space that anesthetists are guiding needles into is an extremely small area, oftentimes comprising a mere 2-5 mm thick region that the needle must be guided into before the spinal cord is punctured. When using traditional methods, these trained anesthetists are still operating under blind conditions, which increases the chances of harm to the patient in the event of the anesthetist puncturing too far into the epidural space and entering into the spinal cord leading to significant health complications for the patient. There is therefore a need to develop systems and methods to address the above deficiencies.

### SUMMARY

In some embodiments, a measurement system for ultrasonic ranging is provided. The measurement system may include a fiber having at least two cores suitable for guiding light. The measurement system may include an acoustic emitter on a distal end of a first core of the fiber. The acoustic emitter may generate acoustic waves in response to incident light pulses. The measurement system may include an acoustic sensor on a distal end of a second core of the fiber.

In some embodiments, the acoustic emitter may include an absorptive coating.

In some embodiments, the acoustic emitter may include absorptive structures.

In some embodiments, the absorptive structures may include at least one of a coating, microstructures, or nanostructures.

In some embodiments, the acoustic sensor may include a resonator. The acoustic sensor may be configured to detect the acoustic waves by receiving probe light tuned to an optical resonance of the resonator and measuring changes in return light from the resonator.

In some embodiments, the resonator may include at least one of a Fabry-Perot resonator or a spherical resonator.

In some embodiments, the fiber may be a two-core fiber.

In some embodiments, the fiber may be a multicore fiber having more than two cores.

In some embodiments, one or more additional cores of the multicore fiber may be adapted for generating shockwaves that are detectable by the acoustic sensor.

In some embodiments, photoacoustic imaging may be performed by illuminating any of the one or more additional cores and probing the acoustic sensor through the second core.

In some embodiments, the measurement system may be positioned at least partially within a needle.

In some embodiments, the needle may be an epidural needle or a spinal needle.

In some embodiments, the measurement system may further include a control unit coupled to the measurement system.

In some embodiments, the control unit may include one or more light sources to provide input beams for ranging.

In some embodiments, the control unit may include a display to show data associated with ranging.

In some embodiments, the display may include a tablet, a virtual reality (VR) headset, or a monitor.

In some embodiments, ranging may be performed by illuminating the acoustic emitter through the first core to generate an acoustic signal and probing the acoustic sensor through the second core.

In some embodiments, a method for ultrasonic ranging is provided. The method may include propagating light pulses through a first core of a fiber to illuminate an acoustic emitter on a distal end of the first core. The acoustic emitter may generate acoustic waves in response to the light pulses. The method may include detecting reflections of the acoustic waves with an acoustic sensor on a distal end of a second core of the fiber. The method may include probing the acoustic sensor by illuminating the acoustic sensor through the second core and monitoring return light to generate ranging data.

In some embodiments, the fiber may be positioned at least partially within a needle.

In some embodiments, the method may further include displaying data associated with the ranging data on a display of a control unit coupled to the fiber.

In some embodiments, a measurement system is provided. The measurement system may include a gradient index (GRIN) lens. The measurement system may include an acoustic emitter fabricated over a first portion of a face of the GRIN lens. The acoustic emitter may generate acoustic waves in response to incident light pulses. The measurement system may include an acoustic sensor fabricated over a second portion of the face of the GRIN lens.

In some embodiments, the acoustic emitter may be fabricated over a first half of a distal face of the GRIN lens and the acoustic sensor may be fabricated over a second half of the distal face of the GRIN lens.

In some embodiments, the acoustic emitter and the acoustic sensor may collectively occupy less than an entirety of a distal face of the GRIN lens to expose an imaging region of the distal face.

In some embodiments, the measurement system may be configured for both depth ranging and photoacoustic imaging.

In some embodiments, photoacoustic imaging may be performed by scanning an input beam across an input face of the GRIN lens to scan an output beam across tissue via the imaging region. The output beam may induce the tissue to generate an acoustic signal that is measured by probing the acoustic sensor.

In some embodiments, the acoustic emitter may include at least one of an absorptive coating or absorptive structures.

In some embodiments, the absorptive structures may include at least one of microstructures or nanostructures.

In some embodiments, the acoustic sensor may include a resonator. The acoustic sensor may be configured to detect the acoustic waves by receiving probe light tuned to an optical resonance of the resonator and measuring changes in return light from the resonator.

In some embodiments, the resonator may include at least one of a Fabry-Perot resonator or a spherical resonator.

In some embodiments, ranging may be performed by illuminating the acoustic emitter with an input beam at an input face of the GRIN lens to generate acoustic signals and probing the acoustic sensor with another input beam at the input face.

In some embodiments, the GRIN lens may include a multi-pitch configuration.

In some embodiments, the GRIN lens may be fabricated with an undercut pitch providing a focal plane beyond a distal end of the GRIN lens.

In some embodiments, the measurement system may further include an anti-reflective coating applied to at least one end of the GRIN lens.

In some embodiments, the measurement system may further include one or more optical elements configured to manipulate at least one of an intensity distribution or a phase distribution of an input beam to provide a spatially localized output beam from a distal end of the GRIN lens that mitigates aberrations introduced by the GRIN lens.

In some embodiments, the one or more optical elements may include a multi-plane light converter (MPLC) including a mirror and a plurality of phase plates arranged such that the input beam is sequentially modified by the plurality of phase plates.

In some embodiments, the MPLC may be configured to manipulate the intensity distribution and the phase distribution of the input beam to a complex conjugate of a point spread function (PSF) of the GRIN lens such that when a corrected input beam propagates through the GRIN lens, aberrations induced by the GRIN lens are at least partially mitigated.

In some embodiments, the measurement system may further include a demagnification system configured to adjust the corrected input beam to a desired size for input to the GRIN lens.

In some embodiments, the measurement system may further include an input fiber for providing an input beam to the GRIN lens.

In some embodiments, the measurement system may further include one or more actuators for positioning the input fiber to control a position of the input beam on an input face of the GRIN lens.

In some embodiments, the one or more actuators may be remotely placed in a body of a syringe separable from the GRIN lens.

In some embodiments, the syringe and a needle housing the GRIN lens may be couplable using a quick release coupling mechanism.

In some embodiments, the measurement system may be positioned at least partially within a needle.

In some embodiments, the needle may be an epidural needle or a spinal needle.

In some embodiments, the measurement system may further include a medication catheter positioned within the needle.

In some embodiments, the GRIN lens may be positioned within an interior of the medication catheter.

In some embodiments, a measurement system is provided. The measurement system may include a first fiber having an acoustic emitter fabricated on a distal end thereof. The acoustic emitter may generate acoustic waves in response to incident light pulses. The measurement system may include a second fiber having an acoustic sensor fabricated on a distal end thereof. The measurement system may include a third fiber formed as a multicore fiber.

In some embodiments, the acoustic emitter may include at least one of an absorptive coating or absorptive structures.

In some embodiments, the absorptive structures may include at least one of microstructures or nanostructures.

In some embodiments, the acoustic sensor may include a resonator. The acoustic sensor may be configured to detect the acoustic waves by receiving probe light tuned to an optical resonance of the resonator and measuring changes in return light from the resonator.

In some embodiments, the resonator may include at least one of a Fabry-Perot resonator or a spherical resonator.

In some embodiments, ranging may be performed by propagating light through the first fiber to generate an acoustic signal via the acoustic emitter and probing the acoustic sensor through the second fiber.

In some embodiments, photoacoustic imaging may be performed by sequentially propagating an input beam through cores of the third fiber and probing the acoustic sensor through the second fiber.

In some embodiments, a resolution of a generated photoacoustic image may be based on at least one of a size, a layout, or a distribution of cores within the third fiber.

In some embodiments, the measurement system may be positioned at least partially within a needle.

In some embodiments, the needle may be an epidural needle or a spinal needle.

In some embodiments, the measurement system may further include a medication catheter positioned within the needle.

In some embodiments, the measurement system may further include a control unit coupled to the measurement system.

In some embodiments, the control unit may include one or more light sources to provide input beams for at least one of ranging or photoacoustic imaging.

In some embodiments, the control unit may include a display to show data associated with at least one of ranging or photoacoustic imaging.

In some embodiments, the display may include at least one of a tablet, a VR headset, or a monitor.

In some embodiments, a method for ranging and photoacoustic imaging is provided. The method may include propagating light pulses through a first fiber to illuminate an acoustic emitter on a distal end of the first fiber. The acoustic emitter may generate acoustic waves in response to the light pulses. The method may include detecting reflections of the acoustic waves with an acoustic sensor on a distal end of a second fiber to perform ranging. The method may include sequentially propagating input beams through cores of a multicore fiber and detecting resulting acoustic signals with the acoustic sensor to perform photoacoustic imaging.

In some embodiments, the first fiber, the second fiber, and the multicore fiber may be positioned at least partially within a needle during the propagating, detecting, and sequentially propagating.

In some embodiments, the needle may be an epidural needle or a spinal needle.

In some embodiments, the method may further include displaying data associated with at least one of the ranging or the photoacoustic imaging on a display of a control unit.

In some embodiments, the method may further include probing the acoustic sensor by illuminating the acoustic sensor through the second fiber and monitoring return light.

In some embodiments, a measurement system is provided. The measurement system may include a GRIN lens. The measurement system may include an MPLC configured to manipulate an intensity distribution and a phase distribution of an input beam to a complex conjugate of a PSF of the GRIN lens such that when a corrected input beam propagates through the GRIN lens, aberrations induced by the GRIN lens are at least partially mitigated.

In some embodiments, the MPLC may include a mirror and a plurality of phase plates arranged such that the input beam is sequentially modified by the plurality of phase plates.

In some embodiments, the measurement system may further include a demagnification system configured to adjust the corrected input beam to a desired size for input to the GRIN lens.

In some embodiments, the PSF of the GRIN lens may vary as a function of radial position. The MPLC may be configured to provide the corrected input beam having an intensity and phase distribution designed to provide performance within a selected tolerance for a plurality of radial positions.

In some embodiments, the MPLC may be configured based on a PSF corresponding to a non-zero radial position.

In some embodiments, the measurement system may further include an acoustic emitter fabricated over a first portion of a face of the GRIN lens and an acoustic sensor fabricated over a second portion of the face of the GRIN lens.

In some embodiments, the acoustic emitter and the acoustic sensor may collectively occupy less than an entirety of a distal face of the GRIN lens to expose an imaging region of the distal face.

In some embodiments, the acoustic sensor may include a resonator. The acoustic sensor may be configured to detect acoustic waves by receiving probe light tuned to an optical resonance of the resonator and measuring changes in return light from the resonator.

In some embodiments, the resonator may include at least one of a Fabry-Perot resonator or a spherical resonator.

In some embodiments, the measurement system may be configured for both depth ranging and photoacoustic imaging. Depth ranging may be provided by illuminating the acoustic emitter with a first beam and probing the acoustic sensor with a sensing beam. Photoacoustic imaging may be provided by scanning a field of view of the GRIN lens with pulsed excitation light and probing the acoustic sensor with the sensing beam.

In some embodiments, the measurement system may be positioned at least partially within a needle.

In some embodiments, the needle may be an epidural needle or a spinal needle.

In some embodiments, the measurement system may further include a control unit including one or more light sources to provide the input beam.

In some embodiments, the control unit may include a display to show data associated with at least one of ranging or photoacoustic imaging.

In some embodiments, a method for photoacoustic imaging is provided. The method may include providing an input beam to an MPLC to provide a corrected input beam having a modified intensity and phase profile corresponding to a complex conjugate of a PSF of a GRIN lens. The method may include propagating the corrected input beam through the GRIN lens. The method may include providing a spatially-localized output beam from the GRIN lens. Aberrations induced by the GRIN lens may be at least partially mitigated.

In some embodiments, the method may further include demagnifying the corrected input beam prior to propagating the corrected input beam through the GRIN lens.

In some embodiments, the GRIN lens may be positioned at least partially within an epidural needle or a spinal needle during the propagating.

In some embodiments, the method may further include detecting an acoustic signal generated by tissue in response to the spatially-localized output beam.

In some embodiments, the method may further include displaying data associated with the photoacoustic imaging on a display of a control unit.

In some embodiments, the PSF of the GRIN lens may vary as a function of radial position. The MPLC may be configured to provide the corrected input beam having an intensity and phase distribution designed to provide performance within a selected tolerance for a plurality of radial positions.

It is to be understood that both the foregoing general description and the following detailed description are exemplary and explanatory only and are not necessarily restrictive of the invention as claimed. The accompanying drawings, which are incorporated in and constitute a part of the specification, illustrate embodiments of the invention and together with the general description, serve to explain the principles of the invention.

### BRIEF DESCRIPTION OF DRAWINGS

The numerous advantages of the disclosure may be better understood by those skilled in the art by reference to the accompanying figures in which:

FIG. 1 is a block diagram of a measurement system for ultrasonic ranging, in accordance with one or more embodiments of the present disclosure.

FIG. 2A is a cross-sectional view of a measurement system providing ultrasonic ranging, in accordance with one or more embodiments of the present disclosure.

FIG. 2B is a perspective view of a measurement system including a single fiber suitable for both ranging and photoacoustic imaging, in accordance with one or more embodiments of the present disclosure.

FIG. 2C is a perspective view of a measurement system including three fibers, in accordance with one or more embodiments of the present disclosure.

FIG. 3A illustrates a block diagram of a measurement system 100 with a GRIN lens for ultrasonic ranging, in accordance with one or more embodiments of the present disclosure.

FIG. 3B is a side view of a measurement system incorporating a multi-pitch GRIN lens, in accordance with one or more embodiments of the present disclosure.

FIG. 3C is a diagram of a multi-pitch GRIN lens configuration for image relay, in accordance with one or more embodiments of the present disclosure.

FIG. 3D is a diagram of a GRIN lens configured for multi-wavelength operation, in accordance with one or more embodiments of the present disclosure.

FIG. 4A is a cross-sectional view of a GRIN lens in a measurement system suitable for ranging measurements, in accordance with one or more embodiments of the present disclosure.

FIG. 4B is a cross-sectional view of a GRIN lens in a measurement system suitable for both ranging and photoacoustic imaging, in accordance with one or more embodiments of the present disclosure.

FIG. 5 is a perspective view of a multi-plane light converter, in accordance with one or more embodiments of the present disclosure.

FIG. 6 is a diagram depicting the evolution of an intensity profile of an input beam upon interaction with phase plates in a multi-plane light converter configuration, in accordance with one or more embodiments of the present disclosure.

FIG. 7 is a series of plots providing a comparison between a targeted magnified point spread function and an actual multi-plane light converter output, in accordance with one or more embodiments of the present disclosure.

FIG. 8 is a series of simulations showing on-axis aberration correction for a spot at half pitch through a GRIN lens, in accordance with one or more embodiments of the present disclosure.

FIG. 9 is a series of simulations showing off-axis aberration correction for a 25 micrometer off-axis position using a shifted on-axis point spread function, in accordance with one or more embodiments of the present disclosure.

FIG. 10 is a series of simulations showing the impact of off-axis positioning on aberration correction for a 50 micrometer off-axis case, in accordance with one or more embodiments of the present disclosure.

FIG. 11 is a series of simulations illustrating the impact of radial position on aberration correction, in accordance with one or more embodiments of the present disclosure.

FIG. 12 is a plot summarizing a series of experiments for which a multi-plane light converter is designed to correct aberrations for light at different radial values, in accordance with one or more embodiments of the present disclosure.

FIG. 13 is a side view of a measurement system configured for placement within a syringe and needle assembly, in accordance with one or more embodiments of the present disclosure.

FIG. 14A is a cross-sectional view of a co-package parallel configuration for housing components within a needle, in accordance with one or more embodiments of the present disclosure.

FIG. 14B is a cross-sectional view of a co-package coaxial configuration for housing components within a needle, in accordance with one or more embodiments of the present disclosure.

FIG. 15 is a system diagram of a guided needle assembly paired with a control unit, in accordance with one or more embodiments of the present disclosure.

FIG. 16 is a cross-sectional view of a spinal region of a patient showing anatomical structures relevant to epidural needle insertion, in accordance with one or more embodiments of the present disclosure.

FIG. 17 is a flowchart for a method for ultrasonic ranging using a fiber with an acoustic emitter and an acoustic sensor, in accordance with one or more embodiments of the present disclosure.

FIG. 18 is a flowchart for a method for ranging and photoacoustic imaging using a fiber-based measurement system, in accordance with one or more embodiments of the present disclosure.

FIG. 19 is a flowchart for a method for photoacoustic imaging using a multi-plane light converter and gradient index lens, in accordance with one or more embodiments of the present disclosure.

### DETAILED DESCRIPTION

Reference will now be made in detail to the subject matter disclosed, which is illustrated in the accompanying drawings. The present disclosure has been particularly shown and described with respect to certain embodiments and specific features thereof. The embodiments set forth herein are taken to be illustrative rather than limiting. It should be readily apparent to those of ordinary skill in the art that various changes and modifications in form and detail may be made without departing from the spirit and scope of the disclosure.

Reference will now be made in detail to the subject matter disclosed, which is illustrated in the accompanying drawings. The present disclosure has been particularly shown and described with respect to certain embodiments and specific features thereof. The embodiments set forth herein are taken to be illustrative rather than limiting. It should be readily apparent to those of ordinary skill in the art that various changes and modifications in form and detail may be made without departing from the spirit and scope of the disclosure.

Embodiments of the present disclosure are directed to fiber optic systems and methods for depth ranging and/or photoacoustic imaging. In general, the systems and methods disclosed herein utilize acoustic emitters and acoustic sensors positioned at distal ends of optical fibers or lenses to enable ranging and/or imaging capabilities. An acoustic emitter may generate acoustic waves (e.g., ultrasonic waves) in response to incident light pulses, and an acoustic sensor may detect acoustic waves, including reflections of emitted acoustic waves or acoustic signals generated by tissue in response to optical excitation. The acoustic emitter may include any material or component suitable for generating acoustic signals in response to illumination such as, but not limited to, an absorptive coating or absorptive structures. The acoustic sensor may include any component or combination of components suitable for sensing acoustic signals such as, but not limited to, a resonator (e.g., a Fabry-Perot interferometer). For example, acoustic signals may influence a resonator spacing, which may be probed by illuminating the resonator and monitoring return light.

The systems and methods disclosed herein may be suitable for a wide range of applications including, but not limited to, medical procedures such as epidural or spinal injections. For example, systems and methods disclosed herein may facilitate ranging and/or photoacoustic imaging through a needle during a procedure. Furthermore, embodiments of the disclosure provide for depth ranging and imaging components with a comparatively small footprint, which may be placed inside of standard size needles. Even further, embodiments provide for a cost-efficient method of depth ranging and imaging, allowing for a disposable, single-use configuration to alleviate sanitation concerns. However, it is to be understood that these examples are provided solely for illustrative purposes and should not be interpreted as limiting.

In some embodiments, a measurement system includes a fiber having at least two cores suitable for guiding light, with an acoustic emitter on a distal end of a first core and an acoustic sensor on a distal end of a second core. In this configuration, ranging may be performed by illuminating the acoustic emitter through the first core to generate an acoustic signal and probing the acoustic sensor through the second core. The fiber may be a two-core fiber or a multicore fiber having more than two cores. When the fiber is a multicore fiber, one or more additional cores may be adapted for generating shockwaves that are detectable by the acoustic sensor, enabling photoacoustic imaging by illuminating any of the additional cores and probing the acoustic sensor.

In some embodiments, a measurement system includes a gradient index (GRIN) lens with an acoustic emitter fabricated over a first portion of a face of the gradient index lens and an acoustic sensor fabricated over a second portion of the face. The acoustic emitter and acoustic sensor may collectively occupy less than an entirety of a distal face of the gradient index lens to expose an imaging region of the distal face, enabling both depth ranging and photoacoustic imaging. Ranging may be performed by illuminating the acoustic emitter with an input beam at an input face of the gradient index lens to generate acoustic signals and probing the acoustic sensor with another input beam. Photoacoustic imaging may be performed by scanning an input beam across an input face of the gradient index lens to scan an output beam across tissue via the imaging region, wherein the output beam induces the tissue to generate an acoustic signal that is measured by probing the acoustic sensor.

In some embodiments, a measurement system includes three separate fibers: a first fiber having an acoustic emitter fabricated on a distal end thereof, a second fiber having an acoustic sensor fabricated on a distal end thereof, and a third fiber formed as a multicore fiber. In this configuration, ranging may be performed by propagating light through the first fiber to generate an acoustic signal via the acoustic emitter and probing the acoustic sensor through the second fiber. Photoacoustic imaging may be performed by sequentially propagating an input beam through cores of the third fiber and probing the acoustic sensor through the second fiber. A resolution of a generated photoacoustic image may be based on at least one of a size, a layout, or a distribution of cores within the third fiber.

In some embodiments, a measurement system includes a gradient index lens and a multi-plane light converter (MPLC) configured to manipulate an intensity distribution and a phase distribution of an input beam to a complex conjugate of a point spread function of the gradient index lens. When a corrected input beam propagates through the gradient index lens, aberrations induced by the gradient index lens may be at least partially mitigated. The MPLC may include a mirror and a plurality of phase plates arranged such that the input beam is sequentially modified by the plurality of phase plates. A demagnification system may adjust the corrected input beam to a desired size for input to the gradient index lens. The point spread function of the gradient index lens may vary as a function of radial position, and the MPLC may be configured to provide the corrected input beam having an intensity and phase distribution designed to provide performance within a selected tolerance for a plurality of radial positions.

Various combinations of the above configurations are contemplated herein. For example, a measurement system may include a gradient index lens with an acoustic emitter and acoustic sensor on portions of a face thereof, along with an MPLC for aberration correction to enable both depth ranging and photoacoustic imaging with improved image quality.

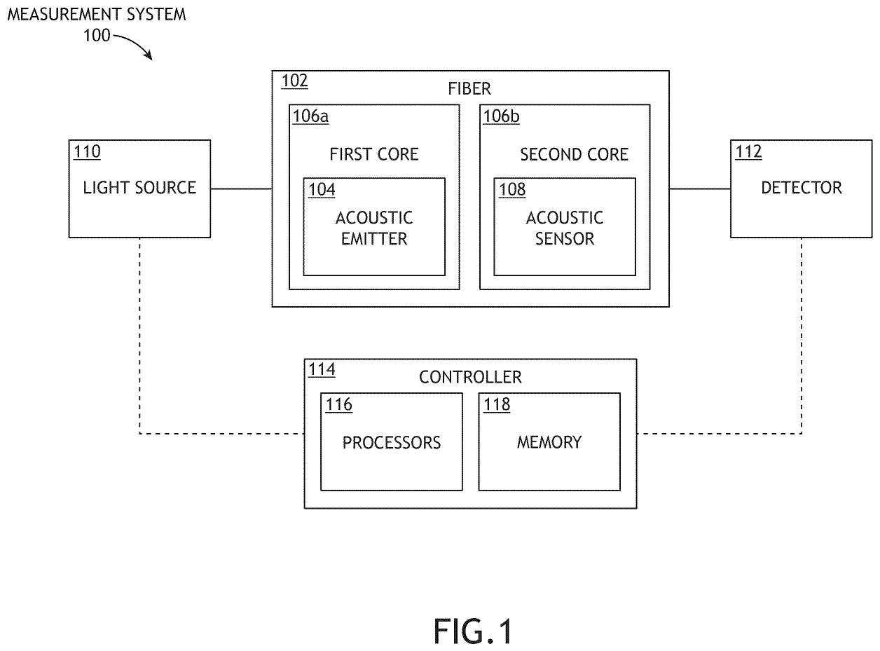

Referring now to FIGS. 1-19, systems and methods providing fiber-based depth ranging and/or photoacoustic imaging are described, in accordance with one or more embodiments of the present disclosure.

FIG. 1 illustrates a block diagram of a measurement system 100 for ultrasonic ranging, in accordance with one or more embodiments of the present disclosure.

In embodiments, a measurement system 100 includes a fiber 102 having at least two cores suitable for guiding light, an acoustic emitter 104 on a distal end of a first core 106a, and an acoustic sensor 108 on a distal end of a second core 106b. The measurement system 100 may further includes one or more light sources 110 coupled to the fiber 102 and configured to provide light for illuminating the acoustic emitter 104 and probing the acoustic sensor 108. The measurement system 100 may further include a detector 112 coupled to the fiber 102 configured to receive return light from the acoustic sensor 108.

In this configuration, the one or more light sources 110 may provide light pulses through the first core 106a to illuminate the acoustic emitter 104, which generates acoustic waves (e.g., ultrasonic waves) in response to the incident light pulses. The acoustic sensor 108 may then be sensitive to reflections of the acoustic waves to provide detection of the acoustic waves. For example, the one or more light sources 110 may provide probe light that propagates through the second core 106b to interact with the acoustic sensor 108 and return to the detector 112, which may generate detection signals representative of the acoustic waves.

The acoustic emitter 104 may include any material or component suitable for generating acoustic signals in response to illumination such as, but not limited to, an absorptive coating or absorptive structures. The absorptive structures may include at least one of a coating, microstructures, or nanostructures. More generally, the acoustic emitter 104 may include any material that absorbs laser energy and generates heat, which in turn produces acoustic waves. Examples of suitable materials include, but are not limited to, carbon-based materials (e.g., carbon nanotubes, graphene, carbon black, or amorphous carbon), light-absorbing dyes, metallic thin films, pigmented polymers, or composite materials incorporating light-absorbing particles.

The acoustic sensor 108 may include any component or combination of components suitable for sensing acoustic waves. In some embodiments, the acoustic sensor 108 includes at least one resonator such as, but not limited to a Fabry-Perot resonator or a spherical resonator. In this configuration, the optical resonance of a resonator may be sensitive to incident acoustic waves such that the presence of the acoustic waves may be characterized by optically probing the acoustic sensor 108 through the second core 106b. For example, the acoustic sensor 108 may be configured to detect the acoustic waves by receiving probe light tuned to an optical resonance of the resonator and measuring changes in return light from the resonator. As another example, acoustic waves may impact a spacing between reflective surfaces of a Fabry-Perot resonator. Accordingly, the measurement system 100 may be configured to detect acoustic waves by propagating light through the second core 106b towards the acoustic sensor 108 and receiving return light with the detector 112.

The detector 112 may include any detector known in the art suitable for measuring optical signals, including but not limited to photodetectors, photodiodes, or spectrometers. Further, any suitable technique for optically probing the acoustic sensor 108 may be utilized. For example, the acoustic sensor 108 may be probed using broadband light, where shifts in the resonance wavelength of a resonator (e.g., a Fabry-Perot resonator) may be detected by monitoring spectral changes in the return light. In this configuration, the detector 112 may include a spectrometer or wavelength-selective detection elements to identify changes in the reflected or transmitted spectrum that correspond to acoustic wave interactions with the resonator. As another example, the acoustic sensor 108 may be probed using narrowband light tuned to a slope of a resonance peak, where acoustic-induced changes in the resonator spacing may modulate the intensity of the return light. In some cases, interferometric techniques may be employed to detect phase changes in the return light caused by acoustic perturbations. The selection of a particular probing technique may depend on factors such as desired sensitivity, bandwidth requirements, or system complexity considerations.

In some embodiments, the measurement system 100 further includes a controller 114 including one or more processors 116 configured to execute program instructions stored in memory 118. The processors 116 of the controller 114 may then execute program instructions causing the processors 116 to implement any of the various steps described in the present disclosure either directly or indirectly (e.g., by generating control signals to control components of the measurement system 100 and/or external components). For example, the processors 116 of the controller 114 may receive signals from the detector 112 indicative of acoustic signals detected by the acoustic sensor 108. As another example, the processors 116 of the controller 114 may generate ranging measurements based on the detected acoustic signals. As another example, the processors 116 of the controller 114 may control the light source 110 to provide light pulses for illuminating the acoustic emitter 104 and probe light for probing the acoustic sensor 108.

The one or more processors 116 of the controller 114 may include any processor or processing element known in the art. For the purposes of the present disclosure, the term “processor” or “processing element” may be broadly defined to encompass any device having one or more processing or logic elements (e.g., one or more micro-processor devices, one or more application specific integrated circuit (ASIC) devices, one or more field programmable gate arrays (FPGAs), or one or more digital signal processors (DSPs)). In this sense, the one or more processors 116 may include any device configured to execute algorithms and/or instructions (e.g., program instructions stored in memory). In some embodiments, the one or more processors 116 may be embodied as a desktop computer, mainframe computer system, workstation, image computer, parallel processor, networked computer, or any other computer system configured to execute a program configured to operate or operate in conjunction with the measurement system 100, as described throughout the present disclosure.

The memory 118 may include any storage medium known in the art suitable for storing program instructions executable by the associated one or more processors 116. For example, the memory 118 may include a non-transitory memory medium. By way of another example, the memory 118 may include, but is not limited to, a read-only memory (ROM), a random-access memory (RAM), a magnetic or optical memory device (e.g., disk), a magnetic tape, a solid-state drive and the like. It is further noted that memory 118 may be housed in a common controller housing with the one or more processors 116. In some embodiments, the memory 118 may be located remotely with respect to the physical location of the one or more processors 116 and controller 114. For instance, the one or more processors 116 of the controller 114 may access a remote memory (e.g., server), accessible through a network (e.g., internet, intranet and the like).

It is contemplated herein that the measurement system 100 may be implemented in a variety of configurations. In some embodiments, portions of the measurement system 100 may be positioned at least partially within a needle such as, but not limited to, an epidural needle or a spinal needle. For example, the fiber 102 may be at least partially positioned within a needle such that a distal end with the acoustic emitter 104 and the acoustic sensor 108 may be placed within the body of a patient during a procedure.

In some embodiments, various components of the measurement system 100 are located in a control unit. For example, a control unit may components such as, but not limited to, one or more light sources 110, one or more detectors 112, and/or a controller 114.

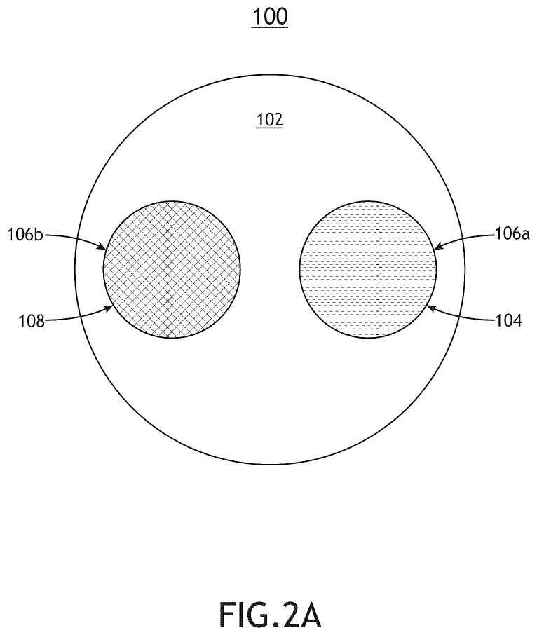

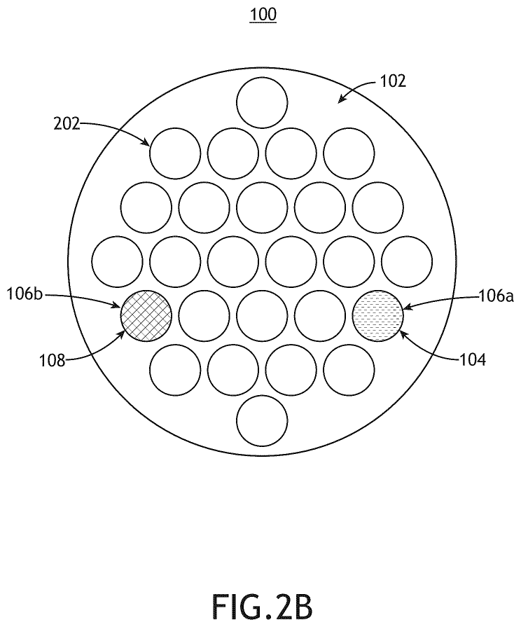

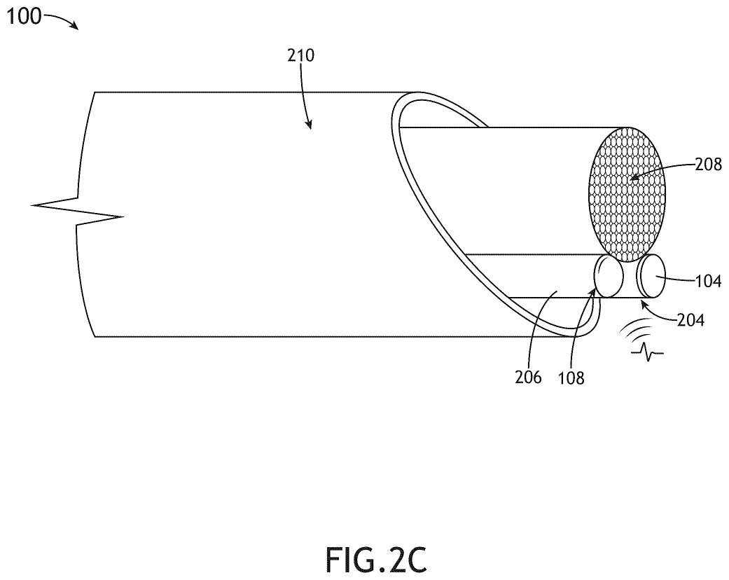

Referring now to FIGS. 2A-2B, the fiber 102 may include any number of cores.

FIG. 2A is a cross-sectional view of a two-core fiber 102 providing ultrasonic ranging, in accordance with one or more embodiments of the present disclosure.

In this configuration, an acoustic emitter 104 may be located on a distal end of a first core 106a and an acoustic sensor 108 may be located on a distal end of a second core 106b. The two-core fiber configuration may provide a compact and cost-effective solution for ranging measurements, as both the acoustic emitter 104 and the acoustic sensor 108 are integrated within a single fiber structure. This arrangement may be particularly suitable for applications where space is limited, such as within medical needles, while maintaining the separation between emission and detection functions to reduce interference and improve measurement accuracy.

FIG. 2B illustrates a cross-sectional view of a multi-core fiber 102 suitable for both ranging and photoacoustic imaging, in accordance with one or more embodiments of the present disclosure.

FIG. 2B is similar to FIG. 2A, where an acoustic emitter 104 is located on a distal end of a first core 106a and an acoustic sensor 108 is located on a distal end of a second core 106b. However, the multi-core fiber 102 in FIG. 2B also includes one or more additional cores 202.

In some embodiments, the one or more additional cores 202 may be adapted for generating shockwaves that are detectable by the acoustic sensor 108. For example, light pulses may be propagated through any of the additional cores 202 to illuminate tissue or other material at a distal end of the fiber 102. The light pulses may induce the tissue or other material to generate shockwaves through photoacoustic excitation, where absorption of the light energy causes localized thermal expansion that produces acoustic signals. These shockwaves may then be detected by the acoustic sensor 108 in a manner similar to that described for ranging applications, such as by probing the acoustic sensor 108 through the second core 106b and monitoring return light with the detector 112. By sequentially propagating light pulses through different ones of the additional cores 202, spatially localized shockwaves may be generated at different locations corresponding to the positions of the respective cores. The acoustic responses from these different locations may be collected and processed (e.g., by the controller 114) to form a photoacoustic image. In this configuration, photoacoustic imaging may be performed by illuminating any of the one or more additional cores 202 and probing the acoustic sensor 108 through the second core 106b. A resolution of the generated photoacoustic image may be based on at least one of a size, a layout, or a distribution of the additional cores 202 within the fiber 102.

FIG. 2C illustrates a perspective view of a measurement system 100 including three separate fibers for ranging and photoacoustic imaging, in accordance with one or more embodiments of the present disclosure.

FIG. 2C is similar to FIG. 2B, except that the acoustic emitter 104 and the acoustic sensor 108 are provided on distal ends of separate fibers rather than cores of a multi-core fiber 102. For example, the acoustic emitter 104 may be located on a distal end of a first fiber 204 and the acoustic sensor 108 may be located on a distal end of a second fiber 206. A third fiber 208 may then be a multi-core fiber.

In this configuration, ranging may be performed by propagating light through the first fiber 204 to generate an acoustic signal via the acoustic emitter 104 and probing the acoustic sensor 108 through the second fiber 206. Photoacoustic imaging may be performed by sequentially propagating an input beam through the various cores of the third fiber 208 to illuminate tissue or other material, which induces the generation of acoustic waves that are detected by probing the acoustic sensor 108 through the second fiber 206.

FIG. 2C further depicts how various fibers (here, the first fiber 204, the second fiber 206, and the third fiber 208) may be partially disposed within a needle 210 for imaging within the body of a patient. More generally, any design of the measurement system 100 including, but not limited to, the designs in FIGS. 2A-2B may include fibers disposed within a needle 210 such as, but not limited to, an epidural needle or a spinal needle, to enable ranging and/or photoacoustic imaging during medical procedures.

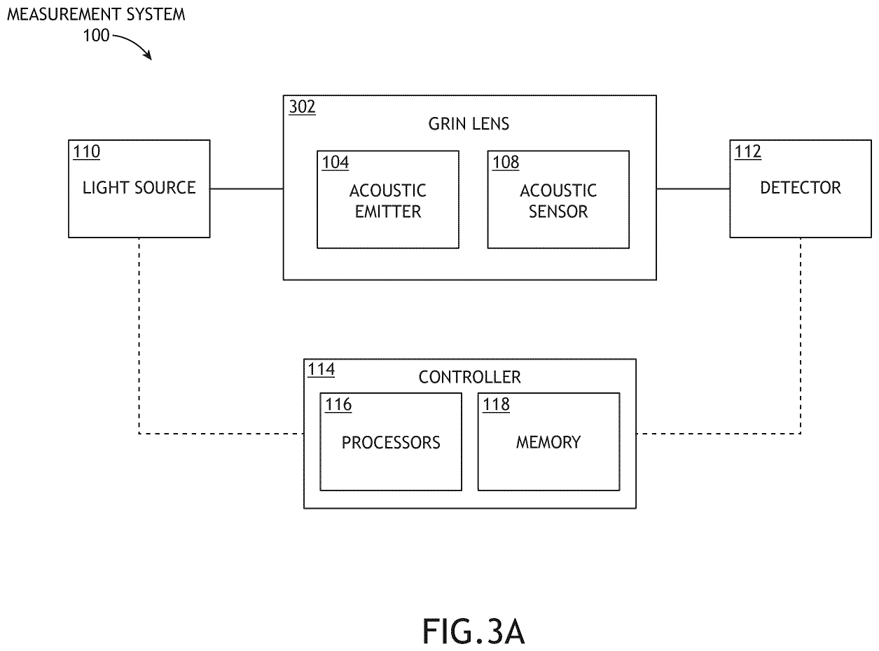

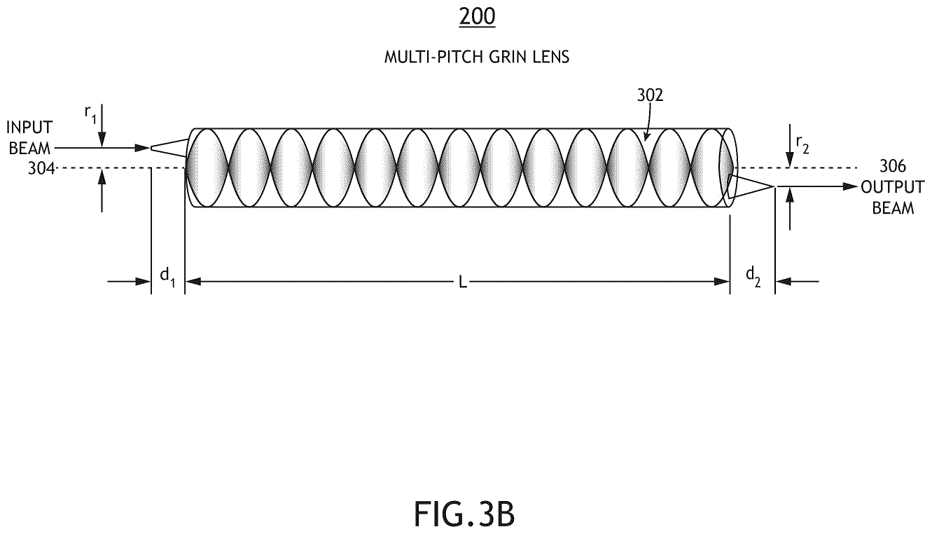

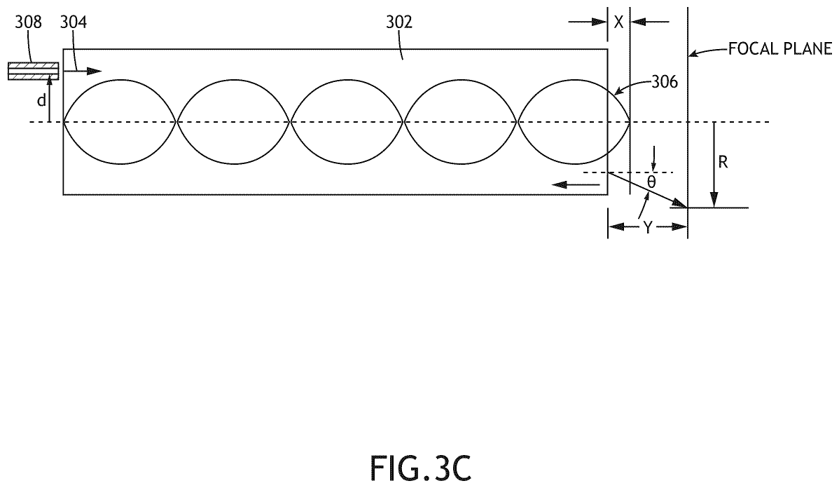

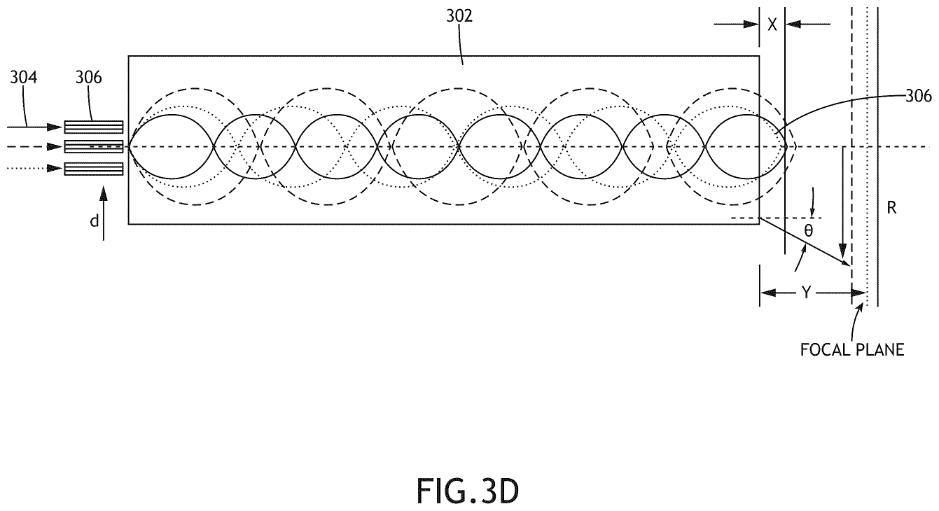

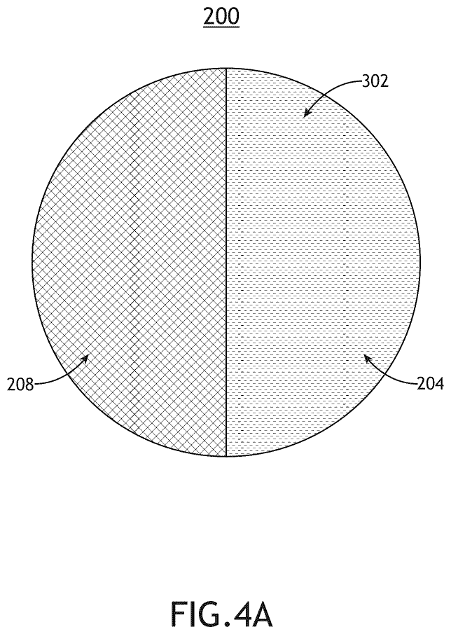

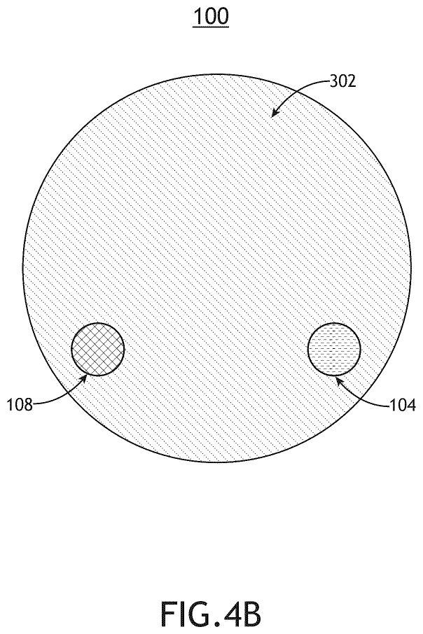

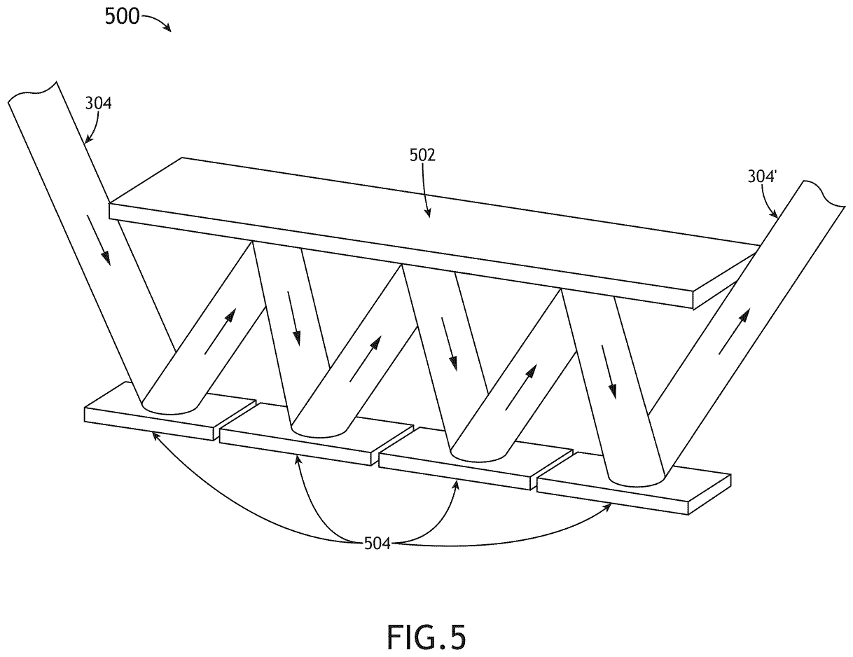

Referring now to FIGS. 3A-5, in some embodiments, the measurement system 100 incorporates at least one GRIN lens, which may be formed as a cylindrically rod-shaped lens with a refractive index that varies in the radial direction. FIGS. 3A-3C depict the operation of a GRIN lens for imaging, FIGS. 4A-4B depict configurations of a GRIN lens 302 within a measurement system 100, and FIG. 5 depicts a GRIN lens in a needle 210.

FIG. 3A illustrates a block diagram of a measurement system 100 with a GRIN lens for ultrasonic ranging, in accordance with one or more embodiments of the present disclosure.

In some embodiments, the measurement system 100 includes a GRIN lens 302 instead of a multi-core fiber 102. In these configurations, the acoustic emitter 104 and the acoustic sensor 108 may be placed on different distal portions of the GRIN lens 302.

It is contemplated herein that the GRIN lens 302 may operate as a lens and may image light from a proximal end to a distal end (e.g., inside an epidural needle). A GRIN lens 302 may comprise a single lens design or alternatively, a multi-lens design, including for example a two-piece GRIN assembly. Further, the GRIN lens 302 may comprise a plurality of pitches, which may be dependent at least on the overall length of the GRIN lens 302. In a GRIN configuration, an image is generated at each half-pitch.

FIG. 3B illustrates a side view of a multi-pitch GRIN lens 302 used for imaging, in accordance with one or more embodiments of the present disclosure.

For example, FIG. 3B depicts a configuration in which a focused input beam 304 located a lateral distance d1 from a proximal end of the GRIN lens 302 and a radial distance r1 from a center optical axis of the GRIN lens 302 is imaged by the GRIN lens 302 to provide an output beam 306 that is focused to a point at a lateral distance d2 from the distal end of the GRIN lens 302 and a radial distance r2. The GRIN lens 302 may provide any focused spot size. For example, spot sizes of 20 microns or below are possible, but not required. In some applications, a multi-pitch GRIN lens 302 with a length of 60 cm and an undercot X of 0.1 pitch may provide a working field of Y as shown in FIG. 3B.

FIGS. 3C and 3D provide additional depictions of imaging with a GRIN lens 302, in accordance with one or more embodiments of the present disclosure.

FIG. 3C illustrates a side view of a multi-pitch GRIN lens 302 with an undercut, in accordance with one or more embodiments of the present disclosure. In FIG. 3C, a length of the GRIN lens 302 is shown with an undercut X, which may provide focusing at an illustrated focal plane with a field of view Y. Further, FIG. 3C depicts a nonlimiting case in which an input beam 304 is provided through a fiber 308 (e.g., a single mode fiber). In this configuration, the position of the focused output beam 306 may be controlled by manipulating the radial and/or lateral position of the fiber 308. For example, one or more actuators may be used to control the radial and/or lateral position of the fiber.

In some embodiments, though not shown, the measurement system 100 may include an input fiber 308 with multiple cores located at different positions relative to an input face of the GRIN lens 302. In this configuration, the position of a focused output beam 306 may be determined through selection of the core.

FIG. 3D illustrates a side view of a multi-pitch GRIN lens 302 depicting multi-wavelength operation, in accordance with one or more embodiments of the present disclosure. It is contemplated herein that the focusing and/or imaging characteristics of a GRIN lens 302 may be wavelength-dependent, which is shown by chromatic variations of the focal plane in FIG. 3D. However, the GRIN lens 302 may be designed to provide performance within specified tolerances for multiple wavelengths.

Further, ranging and/or photoacoustic imaging may be performed using any wavelengths or combinations of wavelengths. For example, the input beams 304 associated with addressing the acoustic emitter 104, photoacoustic excitation, and probing the acoustic sensor 108 may be the same or different wavelengths.

FIG. 3D further depicts three possible positions of an input fiber 308, where in this configuration light is inserted into a middle fiber 308 on an optical axis of the GRIN lens 302.

In some embodiments, an anti-reflective coating may be applied to at least one end of the GRIN lens 302 or across any percentage of the surface area of the GRIN lens 302. The anti-reflective coating may be used to, among other things, increase fidelity of the collected beams by the acoustic sensor 108 by limiting interference caused by reactions between the ultrasound waves and the GRIN lens 302.

Referring now to FIGS. 4A-4B, nonlimiting configurations of a measurement system 100 with a GRIN lens 302 that is suitable for ranging and/or photoacoustic imaging are described, in accordance with one or more embodiments of the present disclosure.

FIG. 4A illustrates a cross-sectional view of a GRIN lens 302 in a measurement system 200 suitable for only ranging measurements, in accordance with one or more embodiments of the present disclosure. In FIG. 4A, an acoustic emitter 104 is fabricated over a first portion (e.g., a first half or any suitable percentage) of a distal face of the GRIN lens 302 and an acoustic sensor 108 is fabricated over a second portion (e.g., a second half or any suitable percentage) of the distal face of the GRIN lens 302. Such a fabrication may operate in a similar manner as the two-core configuration of FIG. 2A, but with a GRIN lens 302.

In some embodiments, a measurement system 200 includes a GRIN lens 302 suitable for providing both ranging and photoacoustic imaging.

FIG. 4B illustrates a cross-sectional view of a GRIN lens 302 in a measurement system 200 suitable for both ranging and photoacoustic imaging, in accordance with one or more embodiments of the present disclosure. In some embodiments, an acoustic emitter 104 is fabricated over a portion of a face of a GRIN lens 302 (e.g., a distal end that may be housed within an epidural needle) and an acoustic sensor 108 is formed over a different portion of the GRIN lens 302. In this configuration, ranging may be performed by illuminating the acoustic emitter 104 with an appropriately placed input beam 304 at an input face of the GRIN lens 302 (e.g., at an appropriate radial and/or lateral position) to generate acoustic signals and probing the acoustic sensor 108 with another appropriately placed input beam 304.

Further, photoacoustic imaging may be performed by scanning an input beam 304 across an input face of the GRIN lens 302, which may scan an output beam 306 across tissue or other material. In this configuration, the focused output beam 306 may induce the tissue or other material to generate an acoustic signal that may be measured by probing the acoustic sensor 108.

It is contemplated herein that such an image may have a gap associated with a location of the acoustic emitter 104. However, this size of this acoustic emitter 104 may be sufficiently small such that the impact of the gap is negligible. Further, in some embodiments, a wavelength of the input beam 304 used for photoacoustic excitation may be selected such that it passes through the acoustic sensor 108 such that this portion of the field of view may contribute to image formation.

Accordingly, the measurement system 100 in FIG. 4A may operate in a manner similar to FIG. 2B, where the multi-core fiber 102 in FIG. 2B is replaced by the GRIN lens 302. Although not explicitly shown, the acoustic emitter 104 and/or the acoustic sensor 108 may be alternatively fabricated on separate fibers or fiber cores in a manner similar to FIG. 2C.

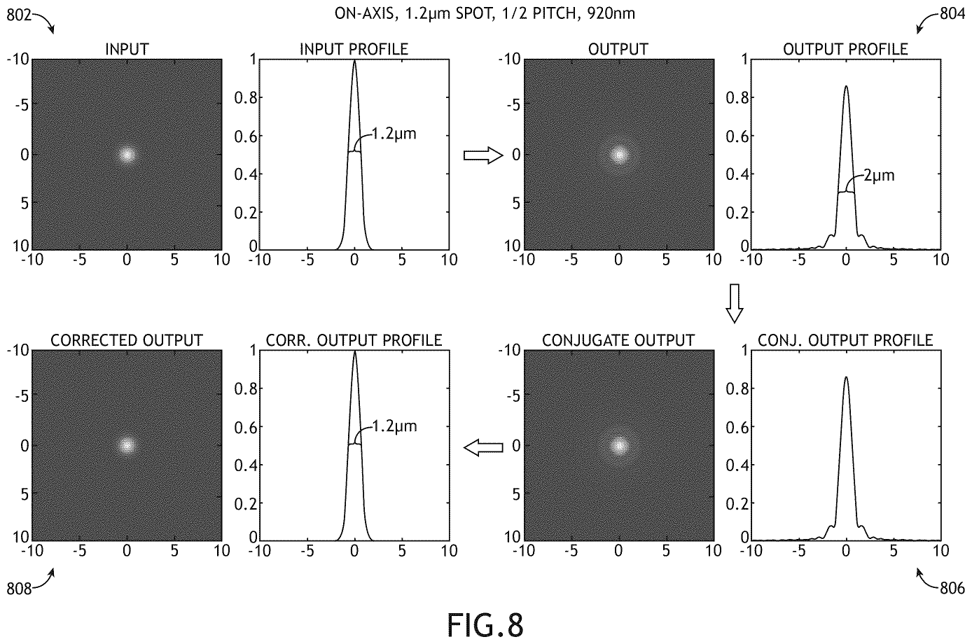

Referring now to FIGS. 8-12, photoacoustic imaging and aberration control using a GRIN lens 302 is described in greater detail, in accordance with one or more embodiments of the present disclosure.

It is contemplated herein that a GRIN lens 302 may introduce aberrations such that an output beam 306 may not have the same distribution (e.g., spot size or shape) as the input beam 304. Further, these aberrations may increase as a size of the GRIN lens 302 increases.

In some embodiments, the measurement system includes a multi-plane light converter (MPLC) to provide aberration correction. In particular, an MPLC may manipulate an intensity and phase distribution of an input beam 304 to a complex conjugate of the point spread function (PSF) of the GRIN lens 302. Thus, when this corrected input beam propagates through the GRIN lens 302, the resulting output beam 306 is at least partially aberration-corrected. In some cases, the output beam 306 may correspond to a point source.

FIG. 5 illustrates a perspective view of a multi-plane light converter (MPLC) 800, in accordance with one or more embodiments of the present disclosure.

In embodiments, the MPLC 500 includes a mirror 502 and a plurality of phase plates 504 arranged such that an input beam 304 is sequentially modified by the phase plates 504. The mirror 502 may extend parallel to the phase plates 504 to facilitate multiple reflections of the input beam 304 and sequential interaction with the phase plates 504. In this way, each phase plate 504 may impart a specific phase modification to the input beam 304 such that the cumulative effect of the sequential interactions with the phase plates 504 provides control over both the intensity and phase distributions of the input beam 304. For example, the phase plates 504 may be designed such that the input beam 304 has an intensity and phase distribution corresponding to a complex conjugate of the point spread function (PSF) of the GRIN lens 302.

After passing through the series of phase plates 504, the corrected input beam 304′ may exit the MPLC 500 and be directed to the GRIN lens. In this way, the corrected input beam 304′ may experience aberrations when propagating through the GRIN lens 302 (e.g., associated with the PSF of the GRIN lens 302). However, since the corrected input beam 304′ is manipulated to have an inverse of the GRIN lens 302 PSF, the resulting output beam 306 is at least partially aberration corrected.

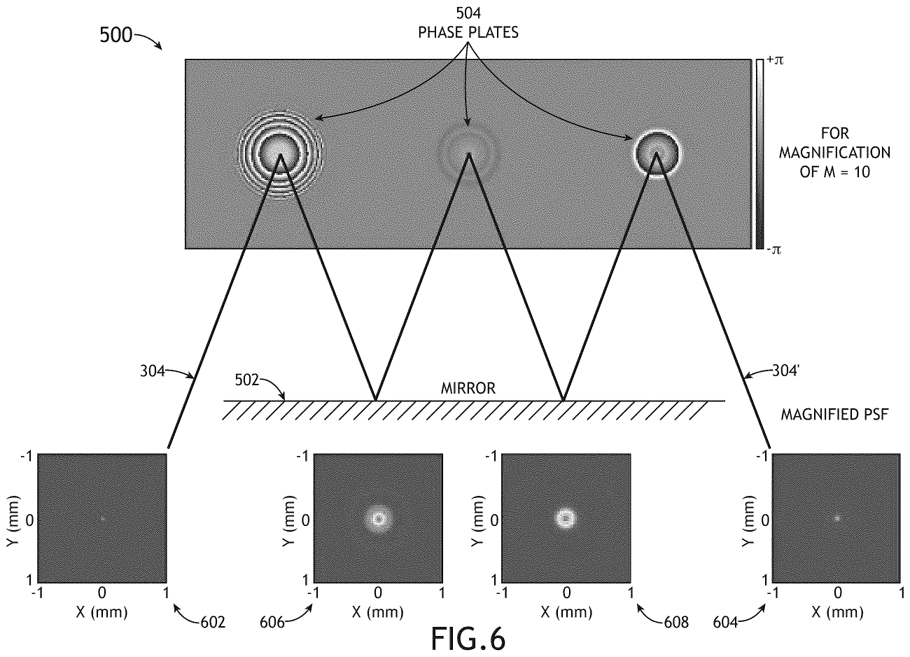

FIG. 6 illustrates the evolution of an intensity profile of an input beam 304 upon interaction with phase plates 504 in an MPLC 500 configuration, in accordance with one or more embodiments of the present disclosure. In this example, the MPLC 500 includes three phase plates 504 designed to provide a corrected input beam 304′ with a desired PSF and also a magnification of 10. Plot 602 depicts an intensity distribution of the input beam 304, plot 604 depicts an intensity distribution of the corrected input beam 304′, and plots 606-608 depict intermediate intensity distributions.

In some embodiments, the measurement system 100 includes an additional magnification (or demagnification) system to further manipulate a size of an input beam 304 entering an MPLC 500 and/or a size of a corrected input beam 304′ from the MPLC 500 entering the GRIN lens 302. In this way, a size of the corrected input beam 304′ entering the GRIN lens 302 may be manipulated without constraint on the MPLC 500.

In some embodiments, the measurement system 100 may include one or more magnification systems and/or demagnification systems positioned on either side of the MPLC 500. For example, a magnification system may be positioned between a light source and the MPLC 500 to expand an input beam 304 to a size suitable for interaction with the phase plates 504, which may allow the phase plates 504 to be fabricated with dimensions and feature resolutions that are practical for manufacturing while still achieving the desired phase modifications. Similarly, a demagnification system may be positioned between the MPLC 500 and the GRIN lens 302 to reduce the size of the corrected input beam 304′ to match the GRIN lens 302. In this way, the phase plates 504 may be designed and fabricated at a convenient scale (e.g., with larger feature sizes that are easier to manufacture), while the corrected input beam 304′ is subsequently adjusted to an appropriate size for coupling into the GRIN lens 302 or other optical elements. The magnification and/or demagnification systems may include any suitable optical components such as, but not limited to, lens assemblies, telescope configurations, or other beam-shaping optics.

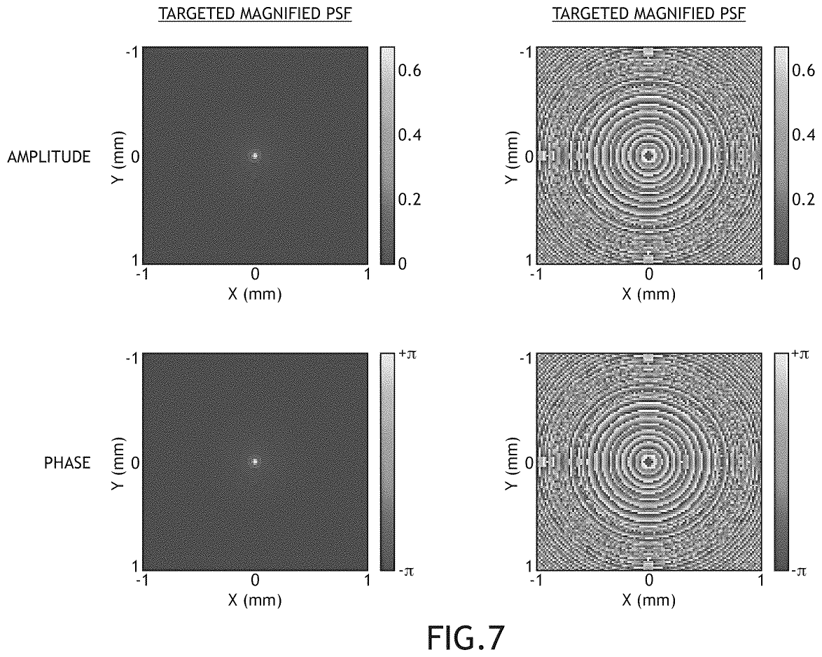

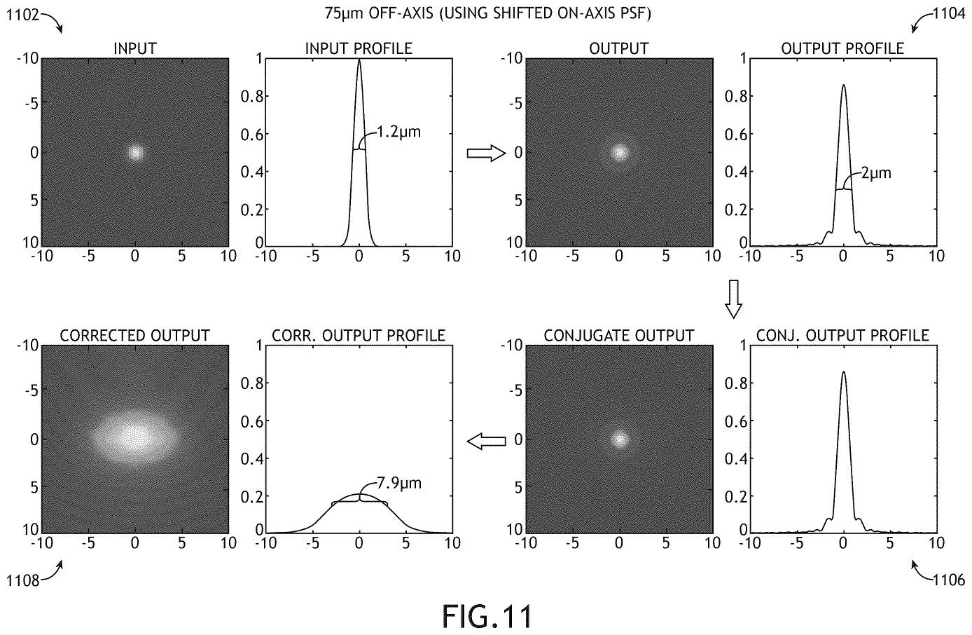

FIG. 7 illustrates a series of plots providing a comparison of the performance of a fabricated MPLC 500 compared to theoretical performance, in accordance with one or more embodiments of the present disclosure. In particular, the left column of FIG. 11 depicts targeted amplitude and phase profiles (e.g., that correspond to a complex conjugate of the PSF of the GRIN lens 302), while the right column depicts actual amplitude and phase profiles. In this case, the absolute value of the overlap integral (known also as the coupling efficiency) between the targeted and actual magnified PSF was found to be 98.63%, which indicates excellent performance.

Referring now to FIGS. 8-11, additional aspects of aberration correction are described, in accordance with one or more embodiments of the present disclosure.

It is contemplated herein that the aberration induced by a GRIN lens 302 (e.g., the PSF of the GRIN lens 302) may vary as a function of radial position (e.g., r1 in FIG. 3A). In this way, the aberration experienced by an input beam 304 as it propagates through the GRIN lens 302 may depend on the radial position at which it enters the GRIN lens 302.

FIGS. 8-11 depict a series of simulations of the impact of radial position on aberration correction. In each of FIGS. 8-11, the simulated GRIN lens 302 has a half-pitch of 920 nm.

FIG. 8 illustrates aberration correction of an on-axis input beam 304, in accordance with one or more embodiments of the present disclosure. Plots 802 depict a 2D and 1 D slice of an intensity distribution of an uncorrected input beam 304 with a Gaussian profile with a 1.2 micron spot size. Plots 804 depict a 2D and 1 D slice of an intensity of an output beam 306 associated with propagation of this uncorrected input beam 304 through a GRIN lens 302, effectively showing the aberrations induced by the GRIN lens 302. In particular, plots 804 show a broadening of the spot size to approximately 2 microns and an associated reduction in peak intensity. Plots 806 depict a 2D and 1 D slice of an intensity distribution corrected input beam 304′ associated with a conjugate of the PSF of the GRIN lens 302, which has the same intensity distribution but a different phase distribution (not shown). For example, the corrected input beam 304′ shown in plots 806 may be generated by an MPLC 500. Plots 808 show a 2D and 1D slice of an intensity distribution of an output beam 306 associated with propagation of the corrected input beam 304′ through the GRIN lens 302. As shown in the plots 808, the aberration pre-correction by the MPLC 500 provides an output beam 306 with approximately the same intensity distribution as an ideal Gaussian beam shown in plots 802.

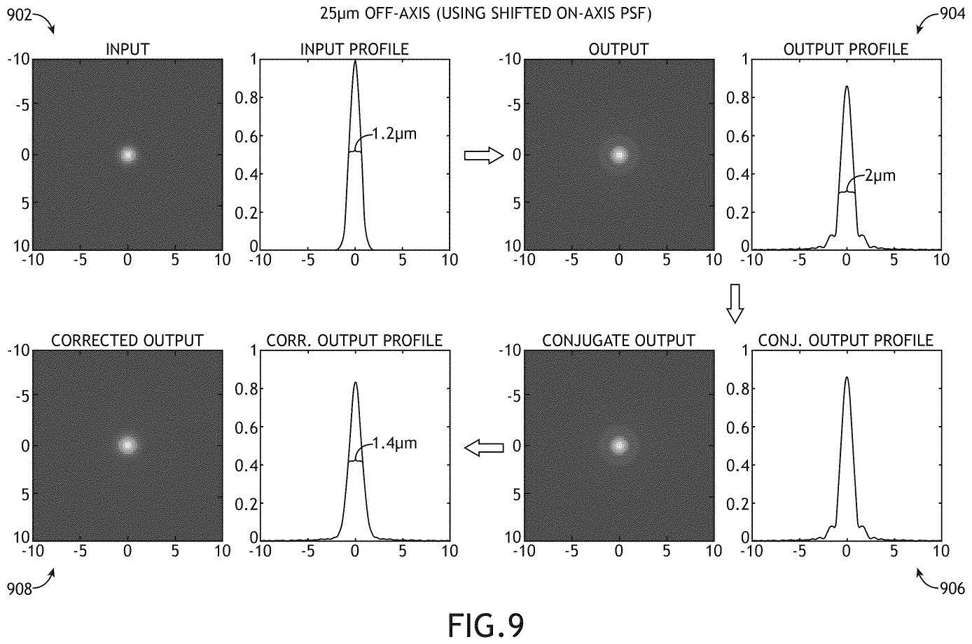

FIG. 9 illustrates aberration correction of an input beam 304 positioned 25 microns off-axis radially, in accordance with one or more embodiments of the present disclosure. Plots 902 depict a 2D and 1 D slice of an intensity distribution of an uncorrected input beam 304 with a Gaussian profile with a 1.2 micron spot size. Plots 904 depict a 2D and 1 D slice of an intensity of an output beam 306 associated with propagation of this uncorrected input beam 304 through a GRIN lens 302, effectively showing the aberrations induced by the GRIN lens 302. In particular, plots 904 show a broadening of the spot size to approximately 2 microns and an associated reduction in peak intensity. Plots 906 depict a 2D and 1 D slice of an intensity distribution corrected input beam 304′ associated with a conjugate of the PSF of the GRIN lens 302, which has the same intensity distribution but a different phase distribution (not shown). For example, the corrected input beam 304′ shown in plots 906 may be generated by an MPLC 500. Plots 908 show a 2D and 1D slice of an intensity distribution of an output beam 306 associated with propagation of the corrected input beam 304′ through the GRIN lens 302. As shown in the plots 908, the aberration pre-correction by the MPLC 500 provides an output beam 306 with a width of approximately 1.4 microns.

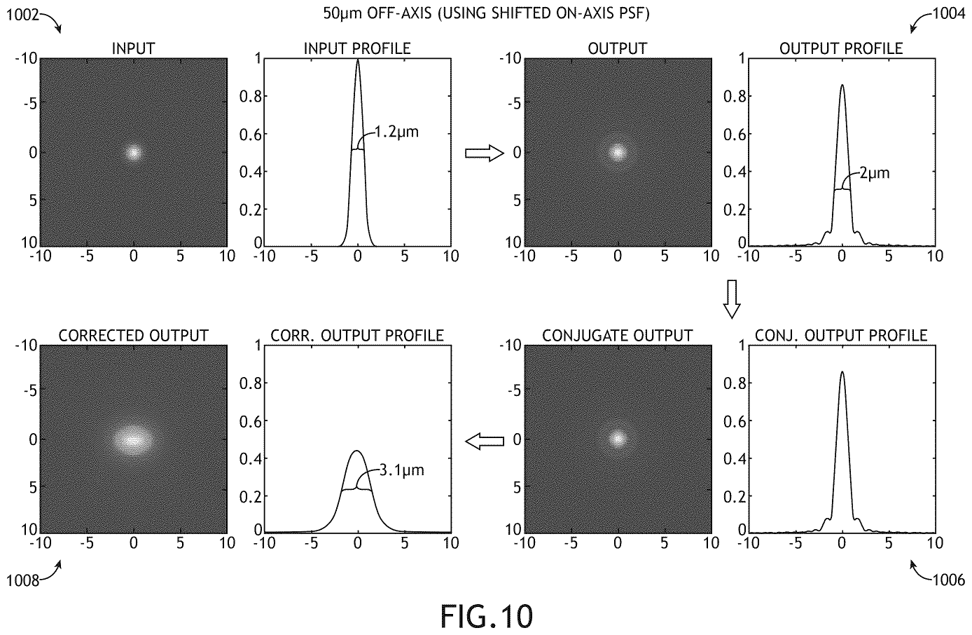

FIG. 10 illustrates aberration correction of an input beam 304 positioned 50 microns off-axis radially, in accordance with one or more embodiments of the present disclosure. Plots 1002 depict a 2D and 1D slice of an intensity distribution of an uncorrected input beam 304 with a Gaussian profile with a 1.2 micron spot size. Plots 1004 depict a 2D and 1 D slice of an intensity of an output beam 306 associated with propagation of this uncorrected input beam 304 through a GRIN lens 302, effectively showing the aberrations induced by the GRIN lens 302. In particular, plots 1004 show a broadening of the spot size to approximately 2 microns and an associated reduction in peak intensity. Plots 1006 depict a 2D and 1 D slice of an intensity distribution corrected input beam 304′ associated with a conjugate of the PSF of the GRIN lens 302, which has the same intensity distribution but a different phase distribution (not shown). For example, the corrected input beam 304′ shown in plots 1006 may be generated by an MPLC 500. Plots 1008 show a 2D and 1D slice of an intensity distribution of an output beam 306 associated with propagation of the corrected input beam 304′ through the GRIN lens 302. As shown in the plots 1008, the aberration pre-correction by the MPLC 500 provides an output beam 306 with a width of approximately 3.1 microns.

FIG. 11 illustrates aberration correction of an input beam 304 positioned 75 microns off-axis radially, in accordance with one or more embodiments of the present disclosure. Plots 1102 depict a 2D and 1D slice of an intensity distribution of an uncorrected input beam 304 with a Gaussian profile with a 1.2 micron spot size. Plots 1104 depict a 2D and 1 D slice of an intensity of an output beam 306 associated with propagation of this uncorrected input beam 304 through a GRIN lens 302, effectively showing the aberrations induced by the GRIN lens 302. In particular, plots 1104 show a broadening of the spot size to approximately 2 microns and an associated reduction in peak intensity. Plots 1106 depict a 2D and 1 D slice of an intensity distribution corrected input beam 304′ associated with a conjugate of the PSF of the GRIN lens 302, which has the same intensity distribution but a different phase distribution (not shown). For example, the corrected input beam 304′ shown in plots 1106 may be generated by an MPLC 500. Plots 1108 show a 2D and 1D slice of an intensity distribution of an output beam 306 associated with propagation of the corrected input beam 304′ through the GRIN lens 302. As shown in the plots 1108, the aberration pre-correction by the MPLC 500 provides an output beam 306 with a width of approximately 7.9 microns.

As shown in FIGS. 8-11, performance is optimal (e.g., the output beam 306 has the smallest beam profile) when the corrected input beam 304 propagates through the GRIN lens 302 starting at the radial position for which the MPLC was designed. Performance then substantially degrades the further the radial position deviates from this design position. For example, the distribution of the output beam 306 in FIG. 11 is substantially degraded relative to the distribution of the output beam 306 in FIG. 8.

TABLE 1
depicts aberration correction performance
for a variety of imaging scenarios.

| | | Image Location (microns) | | | |
|---|---|---|---|---|---|
| Spot Size (um) | | 0 | 25 | 50 | 75 |
| PSF Location | 0 | 1.2 | 1.4 | 3.1 | 7.9 |
| (microns) | 25 | 1.4 | 1.2 | 2.2 | 6.3 |
| | 50 | 2.9 | 2.2 | 1.2 | 3.6 |
| | 75 | 6.7 | 5.6 | 3.6 | 1.2 |

As shown in Table 1, aberration-correction performance is generally best when the PSF selected for the corrected input beam 304′ matches the image location (e.g., the radial position of the input and output light) and degrades when the selected PSF does not match the radial position of the image location.

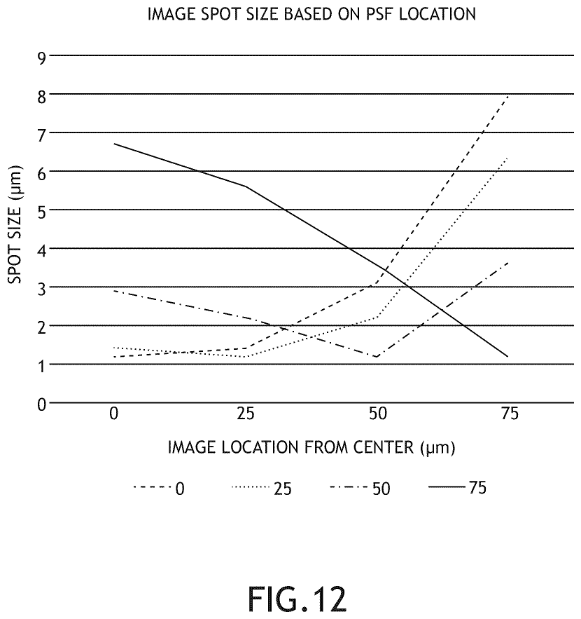

FIG. 12 illustrates a plot depicting image spot size based on PSF location based on the data in Table 1, in accordance with one or more embodiments of the present disclosure.

It is contemplated herein that various techniques for aberration correction may be implemented in a practical system. In some embodiments, the MPLC 500 is adjustable or swappable and is configured to modify the PSF of the corrected input beam 304′ based on the imaging position during photoacoustic imaging. For example, the MPLC 500 may include interchangeable phase plates 504 that can be swapped to provide optimal aberration correction for different radial positions within the field of view. Alternatively, the MPLC 500 may include dynamically adjustable phase plates 504 (e.g., spatial light modulators or deformable mirrors) that can be reconfigured in real-time to match the PSF correction to the current imaging location. As another example, the entire MPLC 500 may be swappable as a modular unit, allowing a user to replace one MPLC 500 configured for a first PSF correction with another MPLC 500 configured for a different PSF correction based on the desired imaging characteristics or radial position range. In this configuration, as the input beam 304 is scanned across different radial positions of the GRIN lens 302, the MPLC 500 may be adjusted to provide the corresponding PSF correction for each position, thereby maintaining optimal aberration correction throughout the imaging process.

In some embodiments, the MPLC 500 is not adjustable and is configured to provide a fixed PSF that provides balanced aberration correction across a range of imaging locations. For example, selecting a PSF associated with a mid-range image location (e.g., r1=50 micrometers as shown in Table 1 or any other non-zero or non-axial location) may provide acceptable aberration correction across the entire imaging field of view. In this configuration, the MPLC 500 may be configured based on a point spread function corresponding to a non-zero radial position. The MPLC 500 may be configured to provide the corrected input beam 304′ having an intensity and phase distribution designed to provide performance within a selected tolerance for a plurality of radial positions. In this configuration, the MPLC 500 may be designed and fabricated with fixed phase plates 504 that provide a compromise PSF correction, accepting slightly degraded performance at on-axis and far off-axis positions in exchange for more uniform performance across the entire field of view. Such a fixed MPLC 500 configuration may be advantageous for applications where simplicity, cost, and reliability are prioritized over achieving optimal aberration correction at every imaging location.

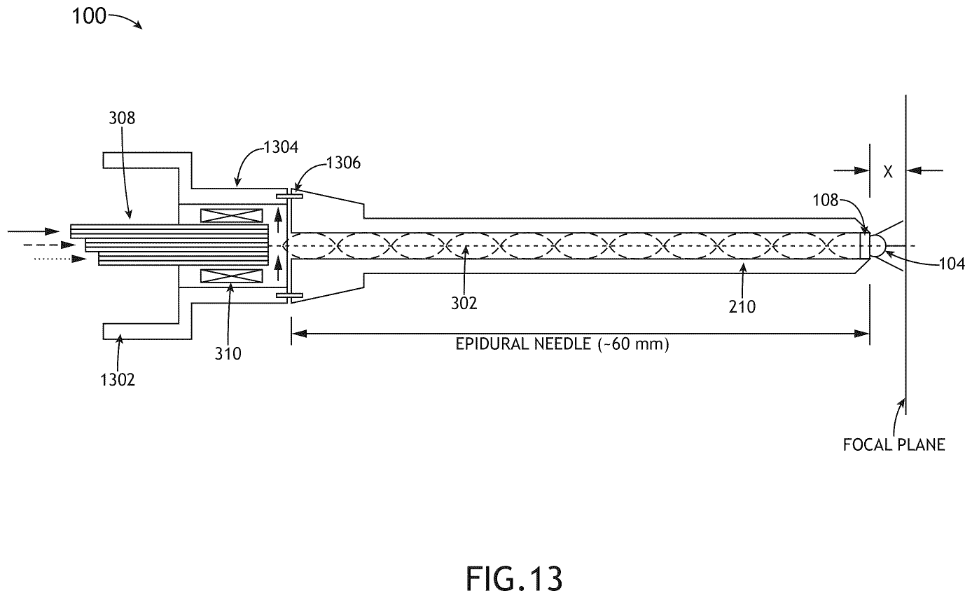

Referring now to FIGS. 13-16, additional aspects of the implementation of the measurement system 100 in a needle 210 are described in greater detail, in accordance with one or more embodiments of the present disclosure.

FIG. 13 illustrates a side view of a measurement system 100 configured for placement within a syringe and needle assembly, in accordance with one or more embodiments of the present disclosure. In this configuration, the measurement system 100 includes a first body component 1302 and a second body component 1304 that may be selectively coupled or decoupled using latching components 1306. The first body component 1302 houses input fibers 308 and actuators 310 for positioning the input fibers 308, while the second body component 1304 houses a GRIN lens 302 that extends into a needle 210. An acoustic emitter 104 and an acoustic sensor 108 are fabricated on portions of a distal face of the GRIN lens 302 within the needle 210. It is contemplated herein that the configuration shown in FIG. 13 is provided as a nonlimiting example, and the GRIN lens 302 may be replaced with any of the fiber configurations described herein, including multi-core fibers or separate fiber arrangements.

The two-body configuration depicted in FIG. 13 may provide various advantages for certain applications. For example, the actuators 310 and input fibers 308 may be remotely placed in the first body component 1302, which may be separable from the second body component 1304 and needle 210. This arrangement may relax space constraints within the needle 210 and enable a more compact needle design. The latching components 1306 may provide a quick release coupling mechanism that allows the first body component 1302 and the second body component 1304 to be coupled with high precision alignment while also permitting separation when desired.

In some embodiments, the measurement system 100 may alternatively include a single monolithic body component rather than the two-body configuration shown in FIG. 13. In such configurations, the input fibers 308, actuators 310, and fiber components (e.g., the GRIN lens 302 or multi-core fibers) may be integrated within a single housing that is coupled to the needle 210. The selection of a particular body configuration may depend on factors such as manufacturing considerations, desired modularity, or application-specific requirements.

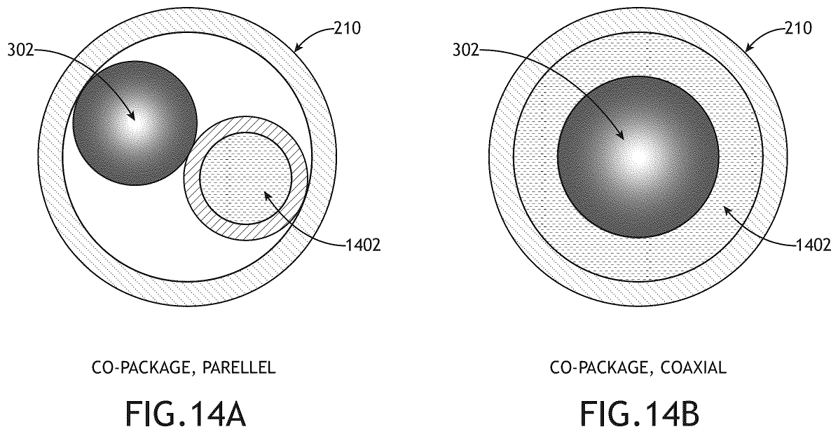

FIGS. 14A and 14B depict exemplary embodiments for the packaging and/or placement of the GRIN lens 302 (or multi-core fiber) while in the epidural needle 210 along with a medication catheter 1402 that may deliver medication to a patient. In particular, FIG. 14A depicts a configuration of the measurement system 100 with a GRIN lens 302 and a medication catheter 1402 placed adjacent to each other within the dimensions of the epidural needle 210. FIG. 14B depicts a second embodiment in which the medication catheter 1402 comprises a larger diameter than the GRIN lens 302 such that the GRIN lens 302 may be placed entirely within the interior of medication catheter 1402.

It is contemplated herein that the configurations shown in FIGS. 14A and 14B are provided solely for illustrative purposes and should not be interpreted as limiting. The measurement system 100 may include any arrangement of optical components, acoustic components, and medication delivery components within the needle 210. For example, the relative positions, orientations, and sizes of the GRIN lens 302 (or any other combination of fibers), medication catheter 1402 may be varied based on factors such as needle dimensions, desired imaging performance, medication delivery requirements, or manufacturing considerations. In some cases, additional components may be included within the needle 210, or certain components may be omitted depending on the intended application.

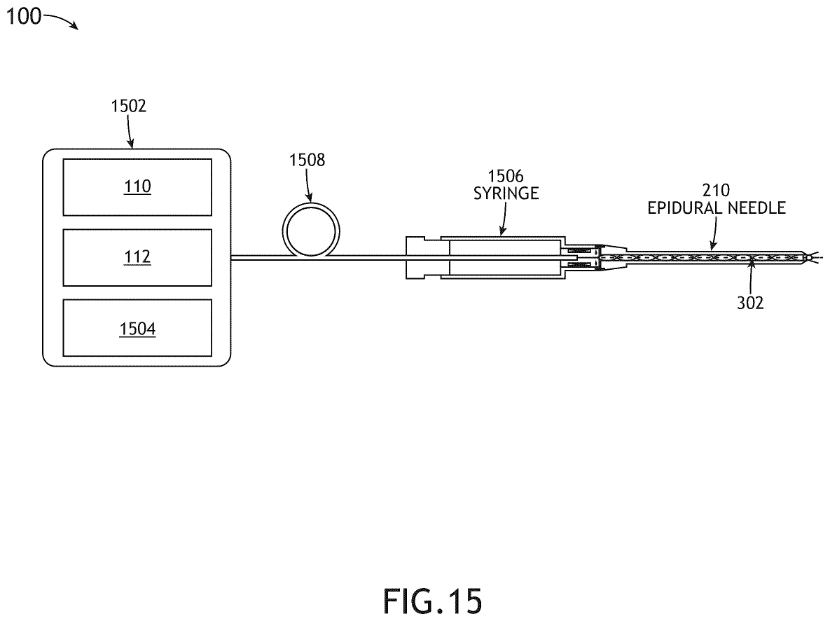

FIG. 15 illustrates a side schematic view of a measurement system 100 including a control unit 1502 couple to a syringe 1506 and an epidural needle 210. In some embodiments, the control unit 1502 includes the one or more light sources 110 and the one or more detectors 112 suitable for ultrasonic ranging and/or photoacoustic imaging as described herein. In some embodiments, the control unit 1502 includes a medication reservoir 1504 for medication to be distributed using the medication catheter 1402 (e.g. using a syringe 1506). The measurement system 100 may further include a coupling 1508 that may include any combination of fiber optics, tubing, or the like suitable for operating the control unit 1502.

In some embodiments, the control unit 1502 includes or is connected to a display to show data associated with ranging and/or photoimaging. The display may be a tablet, a VR headset, or other monitor or display associated with a computing device for displaying ranging and/or image data captured by the measurement system 100, information about the medication catheter 1402, or any other data.

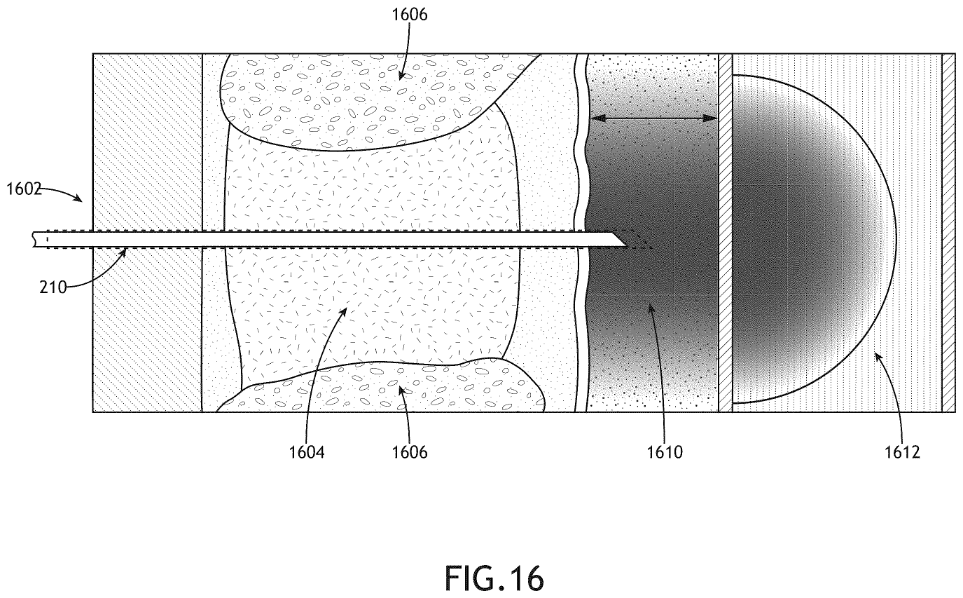

Referring now to FIG. 16, the measurement system 100 may be utilized in any application including, but not limited to, epidural procedures.

FIG. 16 illustrates a cross-section of a spinal region 1602 of a patient into which an epidural needle 210 may be inserted into during a medical procedure, in accordance with one or more embodiments of the present disclosure. In this example, the epidural needle 210 may be inserted into the spinal region 1602 of the patient and first enter through the interspinous ligament 1604 located between two spinous processes 1606. Continuing in this example, the target region for guiding the needle into is epidural space 1610. In a typical medical procedure, the depth of the epidural space 1610 is approximately 2 to 5 mm, with the epidural space 1610 being adjacent to the dura 1612 or spinal cord. In many medical procedures, the epidural needle 210 is guided into the epidural space 1610 for the injection of medication with explicit caution to avoid overextending the epidural needle into the dura 1612, which could lead to significant medical complications for the patient.

Accordingly, embodiments of the measurement system 100 may assist a medical professional in the precise placement of a needle 210 during medical procedures such as epidural or spinal injections. The measurement system 100 may utilize ultrasonic ranging to identify boundaries of the desired epidural space 1610 by generating acoustic waves via the acoustic emitter 104 and detecting reflections of those acoustic waves with the acoustic sensor 108, thereby enabling real-time depth measurements that indicate the distance to tissue interfaces such as the interspinous ligament 1604, the epidural space 1610, and the dura 1612. Simultaneously, the measurement system 100 may perform photoacoustic imaging by propagating light pulses through additional cores of a multicore fiber or by scanning an input beam across a GRIN lens 302 to induce tissue to generate acoustic signals through photoacoustic excitation, with the resulting acoustic signals detected by the acoustic sensor 108 to form images of the surrounding tissue structures. The combination of ranging and photoacoustic imaging capabilities provides the medical professional with comprehensive spatial information, including both depth measurements and structural imaging, to ensure accurate needle placement within the epidural space 1610 while avoiding puncture of the dura 1612 during administration of medication.

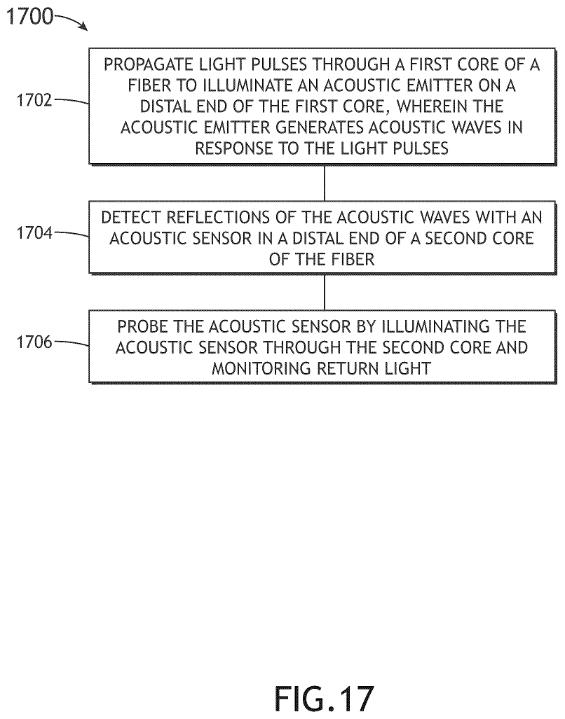

Referring now to FIGS. 17-19, methods for ultrasonic ranging and photoacoustic imaging are described in greater detail, in accordance with one or more embodiments of the present disclosure.

FIG. 17 illustrates a flowchart for a method 1700 for ultrasonic ranging using a fiber with an acoustic emitter and an acoustic sensor, in accordance with one or more embodiments of the present disclosure.

In some embodiments, the method 1700 includes a step 1702 of propagating light pulses through a first core of a fiber to illuminate an acoustic emitter on a distal end of the first core, wherein the acoustic emitter generates acoustic waves in response to the light pulses. Referring to FIGS. 2A and 2B, this step may involve activating one or more light sources 110 to provide light pulses that propagate through the first core 106a of the fiber 102 to reach the acoustic emitter 104. As illustrated in FIG. 2A, the acoustic emitter 104 is located on an end of the first core 106a in a two-core fiber configuration, while FIG. 2B depicts a multicore fiber configuration where the acoustic emitter 104 is fabricated on one or more cores at a distal end of the fiber 102. The acoustic emitter 104 may include absorptive materials such as carbon-based materials, light-absorbing dyes, metallic thin films, or composite materials that absorb the laser energy and generate heat, which in turn produces acoustic waves. The light pulses may have characteristics suitable for efficient acoustic wave generation, including pulse durations and energy levels selected based on the properties of the acoustic emitter 104.

In some embodiments, the method 1700 includes a step 1704 of detecting reflections of the acoustic waves with an acoustic sensor on a distal end of a second core of the fiber. Referring again to FIGS. 2A and 2B, this step may involve the acoustic sensor 108 sensing acoustic waves that have reflected from tissue interfaces or other structures in the vicinity of the distal end of the fiber 102. As shown in FIG. 2A, the acoustic sensor 108 is located on an end of the second core 106b in the two-core fiber 102 configuration, providing a compact arrangement for ranging measurements. In the multicore fiber configuration of FIG. 2B, the acoustic sensor 108 is fabricated on one or more additional cores 202 at the distal end of the fiber 102. The acoustic sensor 108 may include a resonator such as a Fabry-Perot resonator or a spherical resonator, where the optical resonance of the resonator is sensitive to incident acoustic waves. The reflections of the acoustic waves may provide information about the distance to tissue interfaces, enabling depth measurements during medical procedures such as epidural or spinal injections.

In some embodiments, the method 1700 includes a step 1706 of probing the acoustic sensor by illuminating the acoustic sensor through the second core and monitoring return light to generate ranging data. As depicted in FIGS. 2A and 2B, this step may involve the one or more light sources 110 providing probe light that propagates through the second core 106b to interact with the acoustic sensor 108. For example, in the two-core fiber configuration of FIG. 2A, probe light propagates through the second core 106b positioned adjacent to the first core 106a within the fiber 102. In the multicore fiber configuration of FIG. 2B, probe light may propagate through the core or cores associated with the acoustic sensor 108 while the additional cores 202 remain available for photoacoustic imaging applications. The detector 112 may receive return light from the acoustic sensor 108 and generate detection signals representative of the acoustic waves, which may be processed by the controller 114 to generate ranging data indicative of distances to tissue interfaces or other structures. The probing may be performed using broadband light where shifts in the resonance wavelength of a resonator are detected by monitoring spectral changes in the return light, or using narrowband light tuned to a slope of a resonance peak where acoustic-induced changes modulate the intensity of the return light.

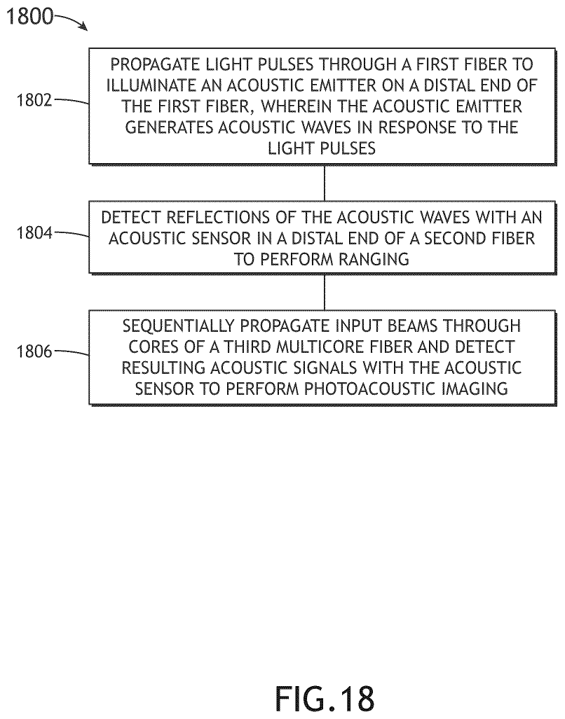

FIG. 18 illustrates a flowchart for a method 1800 for ranging and photoacoustic imaging using a fiber-based measurement system, in accordance with one or more embodiments of the present disclosure.

In some embodiments, the method 1800 includes a step 1802 of propagating light pulses through a first fiber to illuminate an acoustic emitter on a distal end of the first fiber, wherein the acoustic emitter generates acoustic waves in response to the light pulses. Referring to FIGS. 2C and 18, this step may involve the one or more light sources 110 providing light pulses through the first fiber 204 to reach the acoustic emitter 104 fabricated on the distal end thereof. As illustrated in FIG. 2C, the first fiber 204 is positioned within the needle 210 alongside the second fiber 206 and the third fiber 208. The acoustic emitter 104 may generate ultrasonic waves in response to the incident light pulses, with the acoustic waves propagating outward from the distal end of the first fiber 204 toward surrounding tissue or other material.

In some embodiments, the method 1800 includes a step 1804 of detecting reflections of the acoustic waves with an acoustic sensor on a distal end of a second fiber to perform ranging. Referring again to FIGS. 2C and 18, this step may involve the acoustic sensor 108 fabricated on the distal end of the second fiber 206 detecting acoustic waves that have reflected from tissue interfaces. As depicted in FIG. 16, the detected reflections may be used to determine distances to various tissue structures, such as the interspinous ligament 1604, the epidural space 1610, and the dura 1612 during epidural procedures. The controller 114 may process signals from the detector 112 to generate ranging measurements based on the time-of-flight of the acoustic waves.

In some embodiments, the method 1800 includes a step 1806 of sequentially propagating input beams through cores of a third multicore fiber and detecting resulting acoustic signals with the acoustic sensor to perform photoacoustic imaging. Referring to FIGS. 2B, 2C, and 18, this step may involve the one or more light sources 110 sequentially providing input beams through different cores of the third fiber 208 to illuminate tissue or other material at different spatial locations corresponding to the positions of the respective cores. As shown in FIG. 2B, the third fiber 208 includes multiple additional cores 202 arranged in a pattern suitable for photoacoustic imaging. The light pulses may induce the tissue to generate acoustic waves through photoacoustic excitation, where absorption of the light energy causes localized thermal expansion that produces acoustic signals. The acoustic sensor 108 may detect these acoustic signals, and the controller 114 may process the collected acoustic responses to form a photoacoustic image. A resolution of the generated photoacoustic image may be based on at least one of a size, a layout, or a distribution of cores within the third fiber 208.

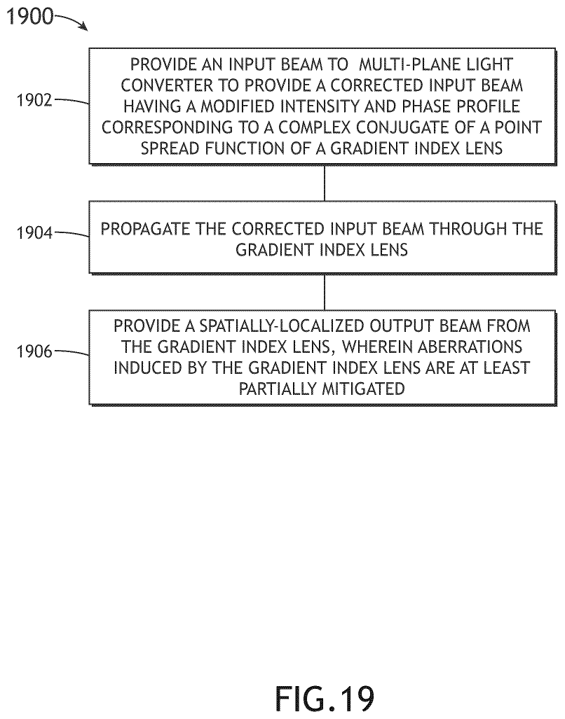

FIG. 19 illustrates a flowchart for a method 1900 for photoacoustic imaging using a multi-plane light converter and gradient index lens, in accordance with one or more embodiments of the present disclosure.

In some embodiments, the method 1900 includes a step 1902 of providing an input beam to a multi-plane light converter to provide a corrected input beam having a modified intensity and phase profile corresponding to a complex conjugate of a point spread function of a gradient index lens. Referring to FIGS. 5, 6, and 19, this step may involve directing an input beam 304 from the one or more light sources 110 into the MPLC 500, where the input beam 304 is sequentially modified by the plurality of phase plates 504. As illustrated in FIG. 5, each phase plate 504 may impart a specific phase modification to the input beam 304 such that the cumulative effect of the sequential interactions provides control over both the intensity and phase distributions. As shown in FIG. 6, the resulting corrected input beam 304′ may have characteristics designed to compensate for aberrations that would otherwise be introduced by the GRIN lens 302.

In some embodiments, the method 1900 includes a step 1904 of propagating the corrected input beam through the gradient index lens. Referring to FIGS. 3A-3C, 5, and 19, this step may involve directing the corrected input beam 304′ from the MPLC 500 into the GRIN lens 302. In some embodiments, the method 1900 includes demagnifying the corrected input beam 304′ prior to propagating the corrected input beam 304′ through the gradient index lens. For example, a demagnification system may adjust the corrected input beam 304′ to a desired size for input to the GRIN lens 302 prior to propagation. As depicted in FIGS. 8-11, the corrected input beam 304′ may experience aberrations when propagating through the GRIN lens 302 associated with the point spread function of the GRIN lens 302.

In some embodiments, the method 1900 includes a step 1906 of providing a spatially-localized output beam from the gradient index lens, wherein aberrations induced by the gradient index lens are at least partially mitigated. Referring to FIGS. 8-12 and 19, this step may result from the pre-compensation provided by the MPLC 500, where the corrected input beam 304′ having an inverse of the GRIN lens 302 point spread function produces an output beam 306 that is at least partially aberration corrected. As shown in FIG. 3C, the spatially-localized output beam 306 may be used for photoacoustic excitation of tissue, inducing the tissue to generate acoustic signals that are detected by the acoustic sensor 108. As demonstrated in FIG. 8, in some cases, the output beam 306 may correspond to a point source suitable for high-resolution photoacoustic imaging.

All of the methods described herein may include storing results of one or more steps of the method embodiments in memory. The results may include any of the results described herein and may be stored in any manner known in the art. The memory may include any memory described herein or any other suitable storage medium known in the art. After the results have been stored, the results can be accessed in the memory and used by any of the method or system embodiments described herein, formatted for display to a user, used by another software module, method, or system, and the like. Furthermore, the results may be stored “permanently,” “semi-permanently, “temporarily,” or for some period of time. For example, the memory may be random-access memory (RAM), and the results may not necessarily persist indefinitely in the memory.

It is further contemplated that each of the embodiments of the method described above may include any other step(s) of any other method(s) described herein. In addition, each of the embodiments of the method described above may be performed by any of the systems described herein.

One skilled in the art will recognize that the herein described components operations, devices, objects, and the discussion accompanying them are used as examples for the sake of conceptual clarity and that various configuration modifications are contemplated. Consequently, as used herein, the specific exemplars set forth and the accompanying discussion are intended to be representative of their more general classes. In general, use of any specific exemplar is intended to be representative of its class, and the non-inclusion of specific components, operations, devices, and objects should not be taken as limiting.

As used herein, directional terms such as “top,” “bottom,” “over,” “under,” “upper,” “upward,” “lower,” “down,” and “downward” are intended to provide relative positions for purposes of description, and are not intended to designate an absolute frame of reference. Various modifications to the described embodiments will be apparent to those with skill in the art, and the general principles defined herein may be applied to other embodiments.

With respect to the use of substantially any plural and/or singular terms herein, those having skill in the art can translate from the plural to the singular and/or from the singular to the plural as is appropriate to the context and/or application. The various singular/plural permutations are not expressly set forth herein for sake of clarity.

The herein described subject matter sometimes illustrates different components contained within, or connected with, other components. It is to be understood that such depicted architectures are merely exemplary, and that in fact many other architectures can be implemented which achieve the same functionality. In a conceptual sense, any arrangement of components to achieve the same functionality is effectively “associated” such that the desired functionality is achieved. Hence, any two components herein combined to achieve a particular functionality can be seen as “associated with” each other such that the desired functionality is achieved, irrespective of architectures or intermedial components. Likewise, any two components so associated can also be viewed as being “connected,” or “coupled,” to each other to achieve the desired functionality, and any two components capable of being so associated can also be viewed as being “couplable,” to each other to achieve the desired functionality. Specific examples of couplable include but are not limited to physically mateable and/or physically interacting components and/or wirelessly interactable and/or wirelessly interacting components and/or logically interacting and/or logically interactable components.

Furthermore, it is to be understood that the invention is defined by the appended claims. It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” and the like). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to inventions containing only one such recitation, even when the same claim includes the introductory phrases “one or more” or “at least one” and indefinite articles such as “a” or “an” (e.g., “a” and/or “an” should typically be interpreted to mean “at least one” or “one or more”); the same holds true for the use of definite articles used to introduce claim recitations. In addition, even if a specific number of an introduced claim recitation is explicitly recited, those skilled in the art will recognize that such recitation should typically be interpreted to mean at least the recited number (e.g., the bare recitation of “two recitations,” without other modifiers, typically means at least two recitations, or two or more recitations). Furthermore, in those instances where a convention analogous to “at least one of A, B, and C, and the like” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, and the like). In those instances where a convention analogous to “at least one of A, B, or C, and the like” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, and the like). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

1. A measurement system for ultrasonic ranging, the measurement system comprising:
a fiber having at least two cores suitable for guiding light;
an acoustic emitter on a distal end of a first core of the fiber, wherein the acoustic emitter generates acoustic waves in response to incident light pulses; and
an acoustic sensor on a distal end of a second core of the fiber.

2. The measurement system of claim 1, wherein the acoustic emitter comprises an absorptive coating.

3. The measurement system of claim 1, wherein the acoustic emitter comprises absorptive structures.

4. The measurement system of claim 3, wherein the absorptive structures comprise at least one of a coating, microstructures, or nanostructures.

5. The measurement system of claim 1, wherein the acoustic sensor comprises a resonator, and wherein the acoustic sensor is configured to detect the acoustic waves by receiving probe light tuned to an optical resonance of the resonator and measuring changes in return light from the resonator.

6. The measurement system of claim 5, wherein the resonator comprises at least one of a Fabry-Perot resonator or a spherical resonator.

7. The measurement system of claim 1, wherein the fiber is a two-core fiber.

8. The measurement system of claim 1, wherein the fiber is a multicore fiber having more than two cores.

9. The measurement system of claim 8, wherein one or more additional cores of the multicore fiber are adapted for generating shockwaves that are detectable by the acoustic sensor.

10. The measurement system of claim 9, wherein photoacoustic imaging is performed by illuminating any of the one or more additional cores and probing the acoustic sensor through the second core.

11. The measurement system of claim 1, wherein the measurement system is positioned at least partially within a needle.

12. The measurement system of claim 11, wherein the needle is an epidural needle or a spinal needle.

13. The measurement system of claim 1, further comprising a control unit coupled to the measurement system.

14. The measurement system of claim 13, wherein the control unit includes one or more light sources to provide input beams for ranging.

15. The measurement system of claim 13, wherein the control unit includes a display to show data associated with ranging.

16. The measurement system of claim 15, wherein the display comprises a tablet, a VR headset, or a monitor.

17. The measurement system of claim 1, wherein ranging is performed by illuminating the acoustic emitter through the first core to generate an acoustic signal and probing the acoustic sensor through the second core.

18. A method for ultrasonic ranging, the method comprising:
propagating light pulses through a first core of a fiber to illuminate an acoustic emitter on a distal end of the first core, wherein the acoustic emitter generates acoustic waves in response to the light pulses;
detecting reflections of the acoustic waves with an acoustic sensor on a distal end of a second core of the fiber; and
probing the acoustic sensor by illuminating the acoustic sensor through the second core and monitoring return light to generate ranging data.

19. The method of claim 18, wherein the fiber is positioned at least partially within a needle.

20. The method of claim 18, further comprising displaying data associated with the ranging data on a display of a control unit coupled to the fiber.

21-85. (canceled)
